(19) United States
(12) Patent Application Publication
(10) Pub. No.: US 2008/0020243 A1
(43) Pub. Date: Jan. 24, 2008

(54) CARBONIC ACID ESTER, PRODUCTION PROCESS THEREFOR, AND MAGNETIC RECORDING MEDIUM

(75) Inventors: Masahiko MORI, Odawara-shi (JP); Daisuke URAZOE, Odawara-shi (JP); Hiroshi HASHIMOTO, Odawara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 11/767,097

(22) Filed: Jun. 22, 2007

(30) Foreign Application Priority Data
Jun. 30, 2006 (JP) 2006-180578

(51) Int. Cl. G11B 5/65; C07C 69/96

(52) U.S. Cl. 428/836.1; 558/260

(57) ABSTRACT

A process for producing a carbonic acid ester is provided, the process comprising a step of synthesizing a saturated alkyl carbonic acid ester represented by Formula (1) so as to give the saturated alkyl carbonic acid ester represented by Formula (1) as a crude product and a step of subjecting the crude product to liquid-liquid extraction using a saturated hydrocarbon solvent and a solvent comprising an organic solvent that is not infinitely miscible with the saturated hydrocarbon solvent so as to give the saturated alkyl carbonic acid ester represented by Formula (1) as a purified product 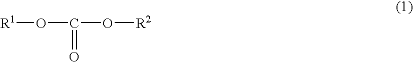
(in Formula (1), R¹ and R² independently denote a saturated hydrocarbon group provided that the sum of the number of carbons in R¹ and the number of carbons in R² is at least 12 but no greater than 50). There are also provided a carbonic acid ester produced by the production process, and a magnetic recording medium comprising a non-magnetic support and, above the non-magnetic support, a magnetic layer comprising a ferromagnetic powder dispersed in a binder, the magnetic layer comprising the carbonic acid ester and having on the surface a number of protrusions that satisfies Formula (2)
0.01≦H₁₅/H₁₀<0.20 (2)
(H₁₀ denotes the number of protrusions per unit area on the surface of the magnetic layer that have a height of less than 10 nm (number/μm²), and H₁₅ denotes the number of protrusions per unit area on the surface of the magnetic layer that have a height of 15 nm or greater (number/μm²)).

### BACKGROUND OF THE INVENTION

1. Field of the Invention

The present invention relates to a production process for a carbonic acid ester that can suitably be used as a lubricant, a carbonic acid ester obtained by the production process, and a magnetic recording medium employing the carbonic acid ester as a lubricant.

2. Description of the Related Art

Magnetic recording technology has the excellent features, not seen in other recording methods, that the medium can be used repeatedly, signals are easily converted to electronic form and it is possible to build a system in combination with peripheral equipment, and signals can easily be corrected, and is therefore widely used in various fields including video, audio, and computer applications.

A magnetic recording medium that satisfies recent requirements for a larger recording capacity and a higher recording density has an extremely smooth surface in order to achieve high electromagnetic conversion characteristics. When a recording head slides against this smooth surface at high speed, it becomes very difficult to ensure durability by conventional techniques.

In order to improve the durability of a magnetic recording medium, for example, a magnetic recording medium employing a carbonate compound as a lubricant has been proposed (JP-A-7-138586 (JP-A denotes a Japanese unexamined patent application publication.) and JP-A-8-77547).

Furthermore, a magnetic recording medium having on the surface a specific abrasive protrusion density and having a specified acid hydrolysis rate has been proposed (JP-A-2003-323711).

### BRIEF SUMMARY OF THE INVENTION

JP-A-7-138586 describes the removal of a by-product originating from a starting material by carrying out distillation during a synthetic process for a carbonate compound, but a high-purity saturated alkyl carbonic acid ester cannot be obtained by the distillation method. When used in, for example, a magnetic recording medium, the presence of a by-product (alcohol, acid, base, etc.) originating from a starting material impairs durability and storage stability, and there is a desire for a method for obtaining a higher-purity saturated alkyl carbonic acid ester.

It is an object of the present invention to provide a production process for a carbonic acid ester, the process enabling a high purity carbonic acid ester to be obtained simply, and to provide a carbonic acid ester obtained by the production process.

It is another object of the present invention to provide a magnetic recording medium having excellent electromagnetic conversion characteristics, durability, and storage stability by the use of the carbonic acid ester.

The objects of the present invention have been attained by means described in (1), (2), or (3).
(1) A process for producing a carbonic acid ester, the process comprising a step of synthesizing a saturated alkyl carbonic acid ester represented by Formula (1) so as to give the saturated alkyl carbonic acid ester represented by Formula (1) as a crude product and a step of subjecting the crude product to liquid-liquid extraction using a saturated hydrocarbon solvent and a solvent comprising an organic solvent that is not infinitely miscible with the saturated hydrocarbon solvent so as to give the saturated alkyl carbonic acid ester represented by Formula (1) as a purified product 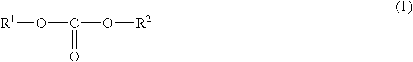
(in Formula (1), R¹ and R² independently denote a saturated hydrocarbon group provided that the sum of the number of carbons in R¹ and the number of carbons in R² is at least 12 but no greater than 50),
(2) a carbonic acid ester produced by the production process according to (1) above, and
(3) a magnetic recording medium comprising a non-magnetic support and, above the non-magnetic support, a magnetic layer comprising a ferromagnetic powder dispersed in a binder, the magnetic layer comprising the carbonic acid ester according to (2) above and having on the surface a number of protrusions that satisfies Formula (2)
0.01≦H₁₅/H₁₀≦0.20 (2)
(H₁₀ denotes the number of protrusions per unit area on the surface of the magnetic layer that have a height of less than 10 nm (number/μm²), and H₁₅ denotes the number of protrusions per unit area on the surface of the magnetic layer that have a height of 15 nm or greater (number/μm²)).

### DETAILED DESCRIPTION OF THE INVENTION

Carbonic Acid Ester and Production Process Therefor

The production process for a carbonic acid ester of the present invention comprises a step of synthesizing a saturated alkyl carbonic acid ester represented by Formula (1) so as to give the saturated alkyl carbonic acid ester represented by Formula (1) as a crude product, and a step of subjecting the crude product to liquid-liquid extraction using a saturated hydrocarbon solvent and a solvent comprising an organic solvent that is not infinitely miscible with the saturated hydrocarbon solvent (hereinafter, also called a ‘polar organic solvent’) so as to give the saturated alkyl carbonic acid ester represented by Formula (1) as a purified product (hereinafter, also called an ‘extraction step’). 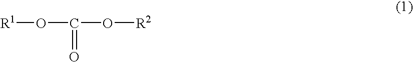
(In Formula (1), R¹ and R² independently denote a saturated hydrocarbon group provided that the sum of the number of carbons in R¹ and the number of carbons in R² is at least 12 but no greater than 50.)

Furthermore, the carbonic acid ester of the present invention is a saturated alkyl carbonic acid ester represented by Formula (1) obtained by the above-mentioned production process, can suitably be used as a lubricant, and can particularly suitably be used as a lubricant used in a magnetic recording medium.

In the present invention, a saturated alkyl carbonic acid ester represented by Formula (1) obtained by the above-mentioned production process is also called ‘a compound of the present invention’, ‘a carbonic acid ester of the present invention’, or ‘a carbonate compound of the present invention’.

It has been found that, when a carbonic acid ester is used in a magnetic recording medium, the carbonic acid ester in the magnetic recording medium causes negative effects such as coloration and crystallization, which are undesirable in practice, due to the presence of an alcohol or a base used as a starting material. As a countermeasure therefor, it has been found that the carbonic acid ester of the present invention can be obtained in high purity by subjecting the carbonic acid ester to liquid-liquid extraction by partitioning the carbonic acid ester into a saturated hydrocarbon solvent such as heptane, and partitioning residues such as an alcohol and a base used as a starting material into a solvent or a mixed solvent that is not infinitely miscible with the saturated hydrocarbon solvent, and preferably into a phase of methanol, acetonitrile, or a mixture thereof.

In Formula (1) above, R¹ and R² are identical or different saturated hydrocarbon groups, and the sum of the number of carbons of the two, that is, R¹ and R², is at least 12 but no greater than 50.

The sum of the number of carbons of the two is preferably 12 to 40, and more preferably 12 to 30. When the sum of the number of carbons of the two is less than 12, the ester is highly volatile, and when it is used as a lubricant in a magnetic recording medium, it vaporizes from the surface of a magnetic layer during transport, thus causing transport failure. When the sum of the number of carbons of the two is greater than 50, the mobility of the ester molecule becomes low, and when it is used as a lubricant in a magnetic recording medium, a necessary amount of lubricant does not exude to the surface, thus causing transport failure.

Furthermore, when producing the carbonic acid ester, if the sum of the number of carbons in R¹ and R² of the carbonic acid ester of the present invention is less than 12, the solubility of the carbonic acid ester in a saturated hydrocarbon solvent becomes poor, which is undesirable in terms of the production process, and if the sum of the number of carbons of the two exceeds 50, the solubility of a residue originating from a starting material, such as an alcohol, in a polar organic solvent becomes poor, which is undesirable in terms of the production process.

Moreover, the saturated hydrocarbon groups denoted by R¹ and R² may be straight or branched chain, and may have a cyclic structure such as cyclohexyl, but they are preferably straight-chain or branched saturated hydrocarbon groups. It is also preferable for either one of R¹ and R² to be straight chain.

Preferred examples of the straight-chain saturated hydrocarbon group include butyl, hexyl, octyl, decyl, dodecyl, tetradecyl, hexadecyl, octadecyl, eicosanyl, and docosanyl.

Preferred examples of the branched saturated hydrocarbon group include 2-butyl, 4-methyl-2-pentyl, 2,2-dimethylpropyl, 2,2-dimethylbutyl, 2-ethylhexyl, 2,2,4,4-tetramethylpentyl, 2-butyloctyl, 2-hexadecyl, and 2-decyltetradecyl.

A process for synthesizing a carbonic acid ester (carbonate) compound represented by Formula (1) of the present invention is not particularly limited, and a known carbonic acid ester synthesis process may be employed. Examples thereof include a process in which a chloroformate ester and an alcohol are reacted, a process in which a carbonic acid ester having a lower hydrocarbon group and an alcohol are reacted, a process in which a diaryl carbonic acid ester and an alcohol are reacted, a process in which carbon monoxide and an alcohol are reacted using a metal catalyst, and a process in which phosgene or a phosgene equivalent such as triphosgene and an alcohol are reacted. Among them, the process in which a chloroformate ester and an alcohol having a saturated hydrocarbon group are reacted is preferable since two different saturated hydrocarbon groups can easily be introduced and a single type of carbonic acid ester can be synthesized. The lower hydrocarbon group referred to here means a hydrocarbon group that has a smaller number of carbons than the saturated hydrocarbon group of the alcohol used in the reaction.

Furthermore, the crude saturated alkyl carbonic acid ester represented by Formula (1) referred to may be a mixture containing a saturated alkyl carbonic acid ester represented by Formula (1) obtained by synthesis, and examples thereof include a reaction solution itself after synthesis of a saturated alkyl carbonic acid ester represented by Formula (1), a filtration product thereof, and a reaction residue obtained by evaporating a solvent from the reaction solution or the filtration product.

Specific examples of the chloroformate ester, which is a starting material for the synthetic reaction, suitably include those that can easily be obtained industrially, such as ethyl chloroformate, butyl chloroformate, sec-butyl chloroformate, isobutyl chloroformate, isopropyl chloroformate, 2-ethylhexyl chloroformate, methyl chloroformate, and propyl chloroformate.

The reaction temperature of the synthetic reaction is not particularly limited as long as the reaction proceeds, but is preferably in the range of 0° C. to 60° C., more preferably 0° C. to 40° C., and yet more preferably 0° C. to 25° C.

The pressure during the synthetic reaction may be a reduced pressure or normal pressure, and normal pressure conditions are preferable from the viewpoint of cost.

The synthetic reaction may employ a catalyst, and when a catalyst is used, it is preferably used at an equivalent amount of 0.001% to 1.0% relative to the carbonate reaction substrate of a chloroformate ester compound, a carbonic acid ester having a lower hydrocarbon group or an aryl group, a phosgene, etc., which are reaction starting materials.

Examples of such a catalyst include organic bases such as pyridine, 4-dimethylaminopyridine, 2-methylpyridine, 4-methylpyridine, imidazole, N-methylimidazole, N-methylmorpholine, and benzotriazole, metal hydroxides such as lithium hydroxide, calcium hydroxide, and magnesium hydroxide, carbonates such as lithium carbonate, sodium carbonate, potassium carbonate, and cesium carbonate, and hydrogen carbonates such as sodium hydrogen carbonate and potassium hydrogen carbonate. Among them, an organic base that does not have an N-H bond when it is neutral, such as pyridine, 4-dimethylaminopyridine, 2-methylpyridine, 4-methylpyridine, or N-methylimidazole, or lithium hydroxide is preferable, and pyridine and derivatives thereof such as pyridine, 4-dimethylaminopyridine, 2-methylpyridine, and 4-methylpyridine are more preferable.

In a production process for the carbonic acid ester of the present invention, as a method for taking out the carbonate compound of the present invention from a reaction liquid, a method is used that comprises a step of carrying out liquid-liquid extraction using a saturated hydrocarbon solvent and a solvent comprising an organic solvent that is not infinitely miscible with the saturated hydrocarbon solvent so as to give the saturated alkyl carbonic acid ester represented by Formula (1) as a purified product, and in order to obtain a carbonic acid ester at a higher purity, the extraction step may be carried out a plurality of times, or a separation method such as extraction by another method, distillation, or crystallization may be carried out in combination.

Solvents used in the extraction step are explained below.

Since the saturated alkyl carbonic acid ester of the present invention has a high solubility in a saturated hydrocarbon-based solvent, as solvents used in the extraction step it is important to use a saturated hydrocarbon solvent and, as a solvent that undergoes phase separation from the saturated hydrocarbon solvent, a solvent comprising an organic solvent that is not infinitely miscible with the saturated hydrocarbon solvent.

The saturated hydrocarbon solvent that can be used in the present invention is not particularly limited as long as it can dissolve the saturated alkyl carbonic acid ester of the present invention, but from the viewpoint of ease of handling of the solvent and ease of a separation operation, a saturated hydrocarbon solvent having a boiling point of 35° C. to 220° C. is preferable, heptane, hexane, decane, undecane, dodecane, cyclohexane, or a mixed solvent thereof is more preferable, and heptane or hexane is yet more preferable. Furthermore, the saturated hydrocarbon solvent may be used singly or as a mixture of two or more types in any proportions.

Furthermore, it is necessary for a polar organic solvent used in the extraction step to dissolve impurities, and in order to remove a base, etc. used in the reaction it is preferable to use an organic solvent that is infinitely miscible with water.

Moreover, since an alcohol used as a starting material for the saturated alkyl carbonic acid ester compound of the present invention generally has extremely low solubility in water, there are cases in which it is necessary to remove as an impurity alcohol remaining in the system as an unreacted component, and as a specific polar organic solvent, a solvent comprising methanol, ethanol, propanol, acetonitrile, ethylene glycol and/or propylene glycol is preferable, and a solvent comprising methanol and/or acetonitrile is more preferable.

In addition to use of the above-mentioned solvent on its own, it is possible to use a mixed solvent that can remove by-products and residual impurities from the saturated hydrocarbon solvent reaction system. The mixed solvent may be a solvent comprising a polar solvent, and preferred specific examples thereof include a mixed solvent of methanol and water, a mixed solvent of acetonitrile and water, a mixed solvent of propylene glycol and water, and a mixed solvent of methanol and ethylene glycol.

With regard to the combination of saturated hydrocarbon solvent and polar organic solvent used in the production process for a carbonic acid ester of the present invention, a combination of the above-mentioned preferred saturated hydrocarbon solvent and the above-mentioned preferred polar organic solvent is also preferable, and a combination of heptane or hexane and methanol, acetonitrile, or a mixed solvent containing at least methanol or acetonitrile is particularly preferable.

Preferred specific examples of the combination of saturated hydrocarbon solvent and polar organic solvent include combinations of hexane and methanol, heptane and acetonitrile, decane and methanol, octane and acetonitrile, octane and methanol, and dodecane and acetonitrile, more preferred examples include combinations of hexane and methanol, heptane and methanol, and heptane and acetonitrile, and yet more preferred examples include a combination of hexane and methanol and a combination of heptane and methanol.

The carbonate compound from which impurities have been removed by use of the production process of the present invention has extremely high purity, and even components that are difficult to detect by gas chromatography, etc. are removed.

Magnetic Recording Medium

The magnetic recording medium of the present invention has, above a non-magnetic support, a magnetic layer comprising a ferromagnetic powder dispersed in a binder, the magnetic layer comprising the carbonic acid ester of the present invention and having on the surface a number of protrusions that satisfies Formula (2).
0.01≦H₁₅/H₁₀≦0.20 (2)
(H₁₀ denotes the number of protrusions per unit area on the surface of the magnetic layer that have a height of less than 10 nm (number/μm²), and H₁₅ denotes the number of protrusions per unit area on the surface of the magnetic layer that have a height of 15 nm or greater (number/μm²).)

The magnetic recording medium of the present invention achieves extremely high durability, electromagnetic conversion characteristics, and storage stability compared with a conventional magnetic recording medium.

For example, in JP-A-7-138586 and JP-A-8-77547, a carbonic acid ester is used in Examples, the surface is relatively rough and it is difficult to guarantee adequate electromagnetic conversion characteristics.

Furthermore, as described in JP-A-2003-323711, a certain degree of electromagnetic conversion characteristics and durability can be guaranteed by making surface properties with a specific protrusion density with respect to an abrasive, but in order to satisfy requirements for sufficient durability for a smooth medium it is necessary to use an alkyl carbonic acid ester having excellent lubrication properties. With regard to a fatty acid ester described in JP-A-2003-323711, the durability is insufficient, it is impossible to prevent the fatty acid ester from undergoing a hydrolysis reaction, and the storage stability is insufficient.

As a result of an intensive investigation, it has been found that, in order to obtain the magnetic recording medium of the present invention having high electromagnetic conversion characteristics and durability, it is necessary for Formula (2) above to be satisfied.

It has also been found that, when an alkyl carbonic acid ester having a carbonate framework that is resistant to hydrolysis and having a lower viscosity than expected for its molecular weight is used in a medium for the above-mentioned surface properties, the electromagnetic conversion characteristics, durability, and storage stability requirements are all sufficiently satisfied.

The present inventors have examined in detail the relationship between the height of protrusions present on the surface of the magnetic layer, various types of lubricant, and the electromagnetic conversion characteristics and transport durability. As a result, it has been found that the presence of the carbonic acid ester of the present invention at an appropriate level on the surface makes the head/tape sliding resistance small, thus improving the durability, and since the carbonic acid ester has a structure that is resistant to hydrolysis compared with conventional fatty acid esters, good storage stability can be guaranteed. Furthermore, both the electromagnetic conversion characteristics and the transport durability strongly depend on the height of protrusions from the surface of the magnetic layer; the electromagnetic conversion characteristics can be improved by decreasing high protrusions and forming a large number of low protrusions, and in the magnetic recording medium of the present invention, good electromagnetic conversion characteristics and good transport durability can be obtained at the same time when 0.01≦H₁₅/H₁₀≦0.30 is satisfied, where the number of protrusions per unit area on the surface of the magnetic layer that have a height of less than 10 nm is H₁₀ and the number of protrusions that have a height of 15 nm or greater is H₁₅. It is more preferable that 0.01≦H₁₅/H₁₀≦0.20. When H₁₅/H₁₀ is smaller than this range, that is, there are too few high protrusions, the reliability is degraded because the ability to remove contamination attached to a head is lost, etc. On the other hand, when there are too many high protrusions, the influence of spacing loss is large, and the electromagnetic conversion characteristics are degraded. However, it has also been found that only controlling the height of protrusions on the surface of the magnetic layer cannot guarantee sufficient transport durability.

It is conventionally known that as an effect of a lubricant present on the surface of a magnetic layer, the sliding properties between a head and a tape are closely related to the amount of lubricant on the surface. A lubricant present on the surface of the magnetic layer in a stable state can reduce the sliding resistance between the head and the tape, thus improving the transport durability. Accompanying a recent demand for higher capacity of magnetic recording media, it is necessary to make the magnetic layer thinner; the amount of lubricant contained in the magnetic layer therefore becomes smaller due to the thinner magnetic layer, the lubricant is gradually removed by sliding against a recording/playback head, and due to an insufficient amount of lubricant scraping off might occur, thus causing stoppages, etc. Furthermore, in order to improve magnetic properties, it is necessary to make the surface of the magnetic layer more and more smooth, and because of this a conventional lubricant cannot exhibit a sufficient effect on transport properties, repetitive transport properties, and durability. When the amount of conventional lubricant is small, if the amount of lubricant is increased in order to enhance the lubrication effect, the mechanical strength of the magnetic coating is degraded, the magnetic layer is scraped off, and scraped-off powder might contaminate the transport path, or sufficient repetitive transport durability cannot be obtained.

Conventionally, a mixture of a fatty acid ester such as butyl stearate and a fatty acid such as myristic acid is used. However, when a fatty acid ester and a fatty acid are used, there is the problem that the friction increases during transport under high humidity conditions, and the transport tension of the magnetic tape becomes high.

When a fatty acid is used on its own, it is necessary to use a large amount thereof in order to obtain slipperiness, and in this case there are the defects that the magnetic layer becomes soft, the mechanical strength is degraded, and high speed sliding durability, which corresponds to the relative speed between the tape and the head, becomes poor. When a fatty acid and a fatty acid ester compound are used in combination, the high speed sliding durability becomes good and the tension becomes relatively low, but there is the defect that the transport tension becomes high under high humidity conditions such as 85% RH (relative humidity).

The present inventors have found that good transport durability can be guaranteed by using as a lubricant a carbonic acid ester (carbonate) having a saturated alkyl group represented by Formula (1) above produced by the production process of the present invention. Since the saturated alkyl carbonic acid ester of the present invention has a lower viscosity than expected for its molecular weight, its fluid lubricating properties are high, and its storage stability is high due to its hydrolysis resistance since it is a carbonate and not a fatty acid ester.

Although JP-A-8-77547 discloses a magnetic recording medium employing an unsaturated alkyl carbonic acid ester, since this carbonic acid ester has an unsaturated group, its miscibility with a binder is high. Because of this, even when a lubricant is added to a thin uppermost layer or a single magnetic layer, only a small amount of lubricant exudes to the surface, and in terms of transport durability the lubricant is gradually removed by sliding against a recording/playback head, thus causing the problem that the transport is halted, etc. It is disclosed that, by adding a lubricant to a lower layer having a thickness of 1 to 5 μm, the amount of lubricant in an upper layer is always supplemented so as to compensate for the lack of lubricant, but sufficient durability cannot be obtained by a medium that has a thin lower layer with a thickness of 1 μm or less in order to meet the recent demand for higher density. It has been found that the saturated alkyl carbonic acid ester of the present invention, which has no unsaturated bond, can guarantee a sufficient amount on the surface by appropriately suppressing miscibility with a binder.

Furthermore, as described above, in accordance with the use of the production process of the present invention, impurities are removed from the carbonic acid ester represented by Formula (1) above and it has an extremely high purity, and the electromagnetic conversion characteristics, durability, and storage stability requirements can all be satisfied by the magnetic recording medium employing same.

In order to control the distribution of the height of protrusions on the surface of the magnetic recording medium there are, for example, the methods as described below.

1) Abrasive dispersion binder: in a method in which an abrasive is dispersed in a binder and a solvent in advance and then added to a magnetic solution containing no abrasive, and they are mixed and dispersed to give a magnetic coating solution, or a method in which an abrasive, a binder, and a solvent are dispersed in advance, this is mixed with a separately dispersed magnetic solution containing no abrasive, and they are further dispersed as necessary to give a magnetic coating solution, the miscibility between the binder used for dispersing the abrasive and the binder in the magnetic solution containing no abrasive is increased or decreased. When the miscibility is high, movement of abrasive particles when a magnetic layer is applied and dried can be suppressed, and the height that the abrasive protrudes can be lowered, whereas when the miscibility is low, the height that the abrasive protrudes can be increased.

2) Strong pressure from calender: the surface of the magnetic layer is molded by means of a hard roll such as a metal roll under high pressure and high temperature so as to push high abrasive protrusions into the magnetic layer. The linear pressure is preferably 2,000 to 4,500 N/cm (200 to 450 kg/cm), and more preferably 2,500 to 4,000 N/cm (250 to 400 kg/cm), and the treatment temperature is preferably 70° C. to 110° C., and more preferably 80° C. to 100° C. The treatment speed is preferably 50 to 400 m/min, and more preferably 80 to 300 m/min. When the linear pressure and the treatment temperature are in the above-mentioned ranges, H₁₅/H₁₀ is in an appropriate range, and the transport durability and the electromagnetic conversion characteristics are excellent.

3) Adjustment of binder: when the Tg of the magnetic layer prior to calendering is reduced by adjusting the type and mixing ratio of binders of the magnetic layer, H₁₅/H₁₀ becomes small even when calendering is carried out under the same conditions. Furthermore, when the amount of binder relative to a magnetic substance is decreased to an appropriate level so that dispersion is not impaired, cavities in the magnetic layer prior to calendering increase, and H₁₅/H₁₀ can be made small even when calendering is carried out under the same conditions.

4) Kneading conditions: when preparing a magnetic coating solution, a kneading treatment is normally carried out using a magnetic substance, a binder, and a small amount of solvent by means of a device such as a kneader with a strong shear force. The kneading treatment increases the adsorptive power of the magnetic substance and the binder, thus increasing the degree of packing of the magnetic layer and increasing the strength of the magnetic layer. When kneading is carried out strongly, the degree of packing increases, but cavities in the magnetic layer after coating decrease, calendering becomes difficult, and H₁₅/H₁₀ increases.

Furthermore, depending on the particle size and the dispersion conditions of the magnetic substance and a non-magnetic powder used in a non-magnetic lower layer, powder aggregates might be contained in the magnetic layer and the non-magnetic lower layer. The surface of such a medium has coarse protrusions, and H₁₅/H₁₀ increases.

5) Blade treatment: the magnetic layer is subjected to a polishing treatment by wrapping a magnetic tape around a polishing tape or wrapping it around a rotating roll having a hard powder such as a diamond powder dispersed thereon, thus cutting off the tops of the protrusions of the abrasive.

I. Magnetic Layer

The magnetic layer of the magnetic recording medium of the present invention is a layer comprising the saturated alkyl carbonic acid ester of the present invention and comprising a ferromagnetic powder dispersed in a binder, and is a layer contributing to magnetic recording and playback.

Ferromagnetic Metal Powder

The ferromagnetic powder used in the magnetic recording medium of the present invention is a cobalt-containing ferromagnetic iron oxide or ferromagnetic alloy powder, and the S_{BET} specific surface area is preferably 40 to 80 m²/g, and more preferably 50 to 70 m²/g. The crystallite size is preferably 12 to 25 nm, more preferably 13 to 22 nm, and particularly preferably 14 to 20 nm. The major axis length is preferably 0.05 to 0.25 μm, more preferably 0.07 to 0.2 μm, and particularly preferably 0.08 to 0.15 μm.

Examples of the ferromagnetic powder include yttrium-containing Fe, Fe—Co, Fe—Ni, and Co—Ni—Fe, and the yttrium content in the ferromagnetic powder is preferably 0.5 to 20 atom % as the yttrium atom/Fe atom ratio Y/Fe, and more preferably 5 to 10 atom %. When it is in such a range, the ferromagnetic powder has a high σs value, and since the iron content is appropriate, the magnetic properties are good, and electromagnetic conversion characteristics are excellent. Furthermore, it is also possible for aluminum, silicon, sulfur, scandium, titanium, vanadium, chromium, manganese, copper, zinc, molybdenum, rhodium, palladium, tin, antimony, boron, barium, tantalum, tungsten, rhenium, gold, lead, phosphorus, lanthanum, cerium, praseodymium, neodymium, tellurium, bismuth, etc. to be present at 20 atom % or less relative to 100 atom % of iron. It is also possible for the ferromagnetic metal powder to contain a small amount of water, a hydroxide, or an oxide.

With regard to the magnetic recording medium of the present invention, one example of a process for producing the ferromagnetic powder into which cobalt or yttrium has been introduced is illustrated below. For example, an iron oxyhydroxide obtained by blowing an oxidizing gas into an aqueous suspension in which a ferrous salt and an alkali have been mixed can be used as a starting material. This iron oxyhydroxide is preferably of the α-FeOOH type, and with regard to a production process therefor, there is a first production process in which a ferrous salt is neutralized with an alkali hydroxide to form an aqueous suspension of Fe(OH)₂, and an oxidizing gas is blown into this suspension to give acicular α-FeOOH. There is also a second production process in which a ferrous salt is neutralized with an alkali carbonate to form an aqueous suspension of FeCO₃, and an oxidizing gas is blown into this suspension to give spindle-shaped α-FeOOH. Such an iron oxyhydroxide is preferably obtained by reacting an aqueous solution of a ferrous salt with an aqueous solution of an alkali to give an aqueous solution containing ferrous hydroxide, and then oxidizing this with air, etc. In this case, the aqueous solution of the ferrous salt may contain an Ni salt, a salt of an alkaline earth element such as Ca, Ba, or Sr, a Cr salt, a Zn salt, etc., and by selecting these salts appropriately the particle shape (axial ratio), etc. can be adjusted.

As the ferrous salt, ferrous chloride, ferrous sulfate, etc. are preferable. As the alkali, sodium hydroxide, aqueous ammonia, ammonium carbonate, sodium carbonate, etc. are preferable. With regard to salts that can be present at the same time, chlorides such as nickel chloride, calcium chloride, barium chloride, strontium chloride, chromium chloride, and zinc chloride are preferable. In a case where cobalt is subsequently introduced into the iron, before introducing yttrium, an aqueous solution of a cobalt compound such as cobalt sulfate or cobalt chloride is mixed and stirred with a slurry of the above-mentioned iron oxyhydroxide. After the slurry of iron oxyhydroxide containing cobalt is prepared, an aqueous solution containing a yttrium compound is added to this slurry, and they are stirred and mixed.

Neodymium, samarium, praseodymium, lanthanum, gadolinium, etc. can be introduced into the ferromagnetic powder used in the present invention as well as yttrium. They can be introduced using a chloride such as yttrium chloride, neodymium chloride, samarium chloride, praseodymium chloride, or lanthanum chloride or a nitrate salt such as neodymium nitrate or gadolinium nitrate, and they can be used in a combination of two or more types. The form of the ferromagnetic powder is not particularly limited, but acicular, granular, cubical, grain-shaped, or tabular form, etc. is normally employed. It is particularly preferable to use an acicular ferromagnetic powder.

As the ferromagnetic powder used in the magnetic layer of the present invention, a hexagonal ferrite powder may also be used.

Examples of the hexagonal ferrite include substitution products of barium ferrite, strontium ferrite, lead ferrite, and calcium ferrite, and Co substitution products. More specifically, magnetoplumbite type barium ferrite and strontium ferrite, magnetoplumbite type ferrite with a particle surface coated with a spinel, magnetoplumbite type barium ferrite and strontium ferrite partially containing a spinel phase, etc., can be cited. In addition to the designated atoms, an atom such as Al, Si, S, Sc, Ti, V, Cr, Cu, Y, Mo, Rh, Pd, Ag, Sn, Sb, Te, Ba, Ta, W, Re, Au, Hg, Pb, Bi, La, Ce, Pr, Nd, P, Co, Mn, Zn, Ni, Sr, B, Ge, Nb, or Zr may be included. For example, those to which Co—Ti, Co—Ti—Zr, Co—Ti—Zn, Ni—Ti—Zn, Nb—Zn—Co, Sb—Zn—Co, Nb—Zn, etc. have been added can be used. Characteristic impurities may be included depending on the starting material and the production process.

The particle size is preferably 10 to 200 nm as a hexagonal plate size, and more preferably 20 to 100 nm. When a magnetoresistive head is used for playback, the plate size is preferably 40 nm or less so as to reduce noise. When the plate size is in such a range, stable magnetization can be expected due to suppression of thermal fluctuations, and noise can be reduced.

The tabular ratio (plate size/plate thickness) is preferably 1 to 15, and more preferably 2 to 7. When the tabular ratio is in such a range, adequate orientation can be obtained, there is little inter-particle stacking, and noise can be suppressed. The specific surface area obtained by the BET method (S_{BET}) of a powder having a particle size within this range is usually 10 to 200 m²/g. The specific surface area substantially coincides with the value obtained by calculation using the plate size and the plate thickness.

The crystallite size is preferably 50 to 450 Å (5 to 45 nm), and more preferably 100 to 350 Å (10 to 35 nm). The plate size and the plate thickness distributions are preferably as narrow as possible. Although it is difficult, the distribution can be expressed using a numerical value by randomly measuring 500 particles on a transmission electron microscope (TEM) photograph of the particles. The distribution is not a regular distribution in many cases, but the standard deviation calculated with respect to the average size is σ/average size=0.1 to 2.0. In order to narrow the particle size distribution, the reaction system used for forming the particles is made as homogeneous as possible, and the particles so formed are subjected to a distribution-improving treatment. For example, a method of selectively dissolving ultrafine particles in an acid solution is also known.

The coercive force (Hc) measured for the magnetic substance can be adjusted so as to be on the order of 500 to 5,000 Oe (39.8 to 398 kA/m). A higher Hc is advantageous for high-density recording, but it is restricted by the capacity of the recording head. The coercive force Hc is preferably on the order of 800 to 4,000 Oe (63.7 to 318 kA/m), and more preferably at least 1,500 Oe (119.4 kA/m) but no greater than 3,500 Oe (278.6 kA/m). When the saturation magnetization of the head exceeds 1.4 T, it is preferably 2,000 Oe or higher. The Hc can be controlled by the particle size (plate size, plate thickness), the type and amount of element included, the element replacement sites, the conditions used for the particle formation reaction, etc.

The saturation magnetization (σs) is 40 to 80 emu/g (40 to 80 A·m²/kg). A higher σs is preferable, but there is a tendency for it to become lower when the particles become finer. In order to improve the σs, making a composite of magnetoplumbite ferrite with spinel ferrite, selecting the types of element included and their amount, etc. are well known. It is also possible to use a W type hexagonal ferrite.

When dispersing the magnetic substance, the surface of the magnetic substance can be treated with a material that is compatible with a dispersing medium and the polymer. With regard to a surface-treatment agent, an inorganic or organic compound can be used. Representative examples include oxides and hydroxides of Si, Al, P, etc., and various types of silane coupling agents and various kinds of titanium coupling agents. The amount thereof is preferably 0.1% to 10% based on the magnetic substance.

The pH of the magnetic substance is also important for dispersion. It is usually on the order of 4 to 12, and although the optimum value depends on the dispersing medium and the polymer, it is selected from on the order of 6 to 10 from the viewpoints of chemical stability and storage properties of the medium. The moisture contained in the magnetic substance also influences the dispersion. Although the optimum value depends on the dispersing medium and the polymer, it is preferably 0.01% to 2.0%.

With regard to a production method for the hexagonal ferrite, there is glass crystallization method (1) in which barium oxide, iron oxide, a metal oxide that replaces iron, and boron oxide, etc. as glass forming materials are mixed so as to give a desired ferrite composition, then melted and rapidly cooled to give an amorphous substance, subsequently reheated, then washed and ground to give a barium ferrite crystal powder; hydrothermal reaction method (2) in which a barium ferrite composition metal salt solution is neutralized with an alkali, and after a by-product is removed, it is heated in a liquid phase at 100° C. or higher, then washed, dried and ground to give a barium ferrite crystal powder; co-precipitation method (3) in which a barium ferrite composition metal salt solution is neutralized with an alkali, and after a by-product is removed, it is dried and treated at 1,100° C. or less, and ground to give a barium ferrite crystal powder, etc., but any production method can be used in the present invention.

Furthermore, as a ferromagnetic powder that can be used in the magnetic layer of the magnetic recording medium of the present invention, iron nitride particles may also be used.

Iron nitride particles that can be used in the present invention are a spherical or ellipsoidal iron nitride-based magnetic substance having at least Fe and N as constituent elements. The ‘spherical’ referred to here means particles having a ratio of the maximum length to the minimum length of the particle diameter of at least 1 but less than 2, and the ‘ellipsoidal’ means particles having a ratio of the maximum length to the minimum length of the particle diameter of at least 2 but less than 4.

The iron nitride-based magnetic substance and a production process therefor are explained below.

Spherical or Ellipsoidal Iron Nitride-Based Magnetic Substance Having at Least Fe and N as Constituent Elements

The iron nitride particles desirably contain at least an Fe₁₆N₂ phase and preferably contain no other iron nitride phase. This is because the magnetocrystalline anisotropy of iron nitride (Fe₄N or Fe₃N phase) is on the order of 1×10⁻¹ J/cm³ (1×10⁵ erg/cc) whereas the Fe₁₆N₂ phase has a high magnetocrystalline anisotropy of 2 to 7×10⁻¹ J/cm³ (2 to 7×10⁶ erg/cc). This allows it to maintain a high coercivity when it is made into fine particles.

This high magnetocrystalline anisotropy is due to the crystal structure of the Fe₁₆N₂ phase. The crystal structure is a body-centered tetragonal system in which N atoms are regularly inserted at interstitial positions in octahedral Fe, and it is surmised that the strain caused by the N atoms being inserted into the lattice results in the occurrence of high magnetocrystalline anisotropy. The axis of easy magnetization of the Fe₁₆N₂ phase is the c-axis, which is elongated by nitriding.

The shape of particles containing the Fe₁₆N₂ phase is preferably spherical or ellipsoidal, and more preferably spherical. Among three equivalent directions of α-Fe, which is a cubic crystal, one direction is selected by nitriding and becomes the c-axis (the axis of easy magnetization) and, unlike acicular particles, if the particle shape is spherical, the axis of easy magnetization is not a mixture of a minor axis direction and a major axis direction, and high magnetocrystalline anisotropy can be achieved. When the maximum diameter of one particle is defined as the major axis and the minimum diameter thereof is defined as the minor axis, the average axial ratio of the major axis length to the minor axis length is preferably 1 to 2, and more preferably 1 to 1.5, and the particle size referred to means the major axis length.

The particle size of the Fe₁₆N₂ phase, which is a magnetic substance, is preferably 5 to 50 nm, and more preferably 10 to 30 nm. When the particle size is 5 nm or greater, there is little influence from fluctuations in heat, there is no superparamagnetization, and it can suitably be used in the magnetic recording medium. Furthermore, due to magnetic viscosity there is an appropriate degree of coercivity when carrying out high-speed recording by a head, and recording properties are excellent. On the other hand, when the particle size is 50 nm or less, saturation magnetization can be made small, the coercivity during recording is appropriate, the recording properties are excellent, and when it is applied to a magnetic recording medium, particulate noise can be suppressed.

The particle size distribution is preferably monodisperse. This is because if it is monodisperse the medium noise is generally reduced. The coefficient of variation of the particle size is preferably 20% or less (1% to 20%), more preferably 15% or less (2% to 15%), and yet more preferably 10% or less (2% to 10%).

The ‘coefficient of variation of particle size’ referred to in the present specification means a value obtained by dividing the standard deviation of the particle size distribution for the diameter of corresponding circles by the average particle size. A ‘coefficient of variation of composition’ means, as for the coefficient of variation of particle size, a value obtained by dividing the standard deviation of the composition distribution of iron nanoparticles by the average composition. In the present invention, these values are multiplied by 100 and expressed as %.

The particle size and the coefficient of variation of particle size may be calculated from an arithmetic average particle size obtained by drying diluted iron nanoparticles on a Cu200 mesh with a carbon film affixed thereto and measuring using a particle size profiler (KS-300, Karl Zeiss) a negative taken at 100,000 times by means of a TEM (1200EX, JEOL).

In particles containing the Fe₁₆N₂ phase, the content of nitrogen relative to iron is preferably 1.0 to 20.0 atm %, more preferably 5.0 to 18.0 atm %, and yet more preferably 8.0 to 15.0 atm %. When the content of nitrogen is 1.0 atm % or greater, the amount of Fe₁₆N₂ phase formed is sufficient, and the increase in coercivity caused by the strain due to nitriding is sufficient. When the content of nitrogen is 20.0 atm % or less, the Fe₁₆N₂ phase, which is a metastable phase, does not decompose and turn into another nitride that is a stable phase, and sufficient saturation magnetization can be obtained.

Fine particulate Fe₁₆N₂ phase has poor oxidation stability, and there is a possibility of ignition if there is no surface compound phase. It is therefore preferable to form a core/shell structure having a surface compound layer formed from an oxide, a nitride, or a carbide, and from the viewpoint of oxidation stability, the surface compound layer is preferably an oxide.

The surface compound layer may be formed by gradually oxidizing the Fe₁₆N₂ phase, but it is preferable to employ a surface compound layer containing at least one element selected from a rare earth element, boron, silicon, aluminum, and phosphorus.

The thickness of the surface compound layer is preferably 1 to 5 nm. When the thickness is 1 nm or greater, the oxidation stability is excellent, when it is 5 nm or less, the proportion of the surface compound layer in the magnetic powder is appropriate, and even if the particle size is small, an appropriate amount of saturation magnetization can be maintained.

With regard to the composition of the surface compound layer, the total content of rare earth element, boron, silicon, aluminum, and phosphorus relative to iron is preferable 0.1 to 40.0 atm %, more preferably 1.0 to 30.0 atm %, and yet more preferably 3.0 to 25.0 atm %. When the content of these elements is 0.1 atm % or greater, it is easy to form the surface compound layer, the magnetic anisotropy of the magnetic powder does not decrease, and the oxidation stability is excellent. When the content of these elements is 40.0 atm % or less, an appropriate level of saturation magnetization can be ensured.

The saturation magnetization (σs) of the Fe₁₆N₂ phase is preferably 50 to 150 emu/g (50 to 150 A·m²/kg), and more preferably 70 to 130 emu/g (70 to 130 A·m²/kg). When the saturation magnetization is 150 emu/g or less, the coercivity during recording is appropriate, and it is easy for a recording head to carry out recording. During playback, even when the saturation magnetization is high, an MR head is not saturated, and an increase in output can be expected. On the other hand, when the saturation magnetization is 50 emu/g or greater, sufficient playback output can be obtained.

Furthermore, this magnetic powder preferably has a BET specific surface area (S_{BET}) of 40 to 100 m²/g. When the BET specific surface area is 40 m²/g or greater, the particle size is appropriate, and when it is applied in a magnetic recording medium particulate noise is suppressed, the surface smoothness of the magnetic layer is excellent, and sufficient playback output can be obtained.

Moreover, when the BET specific surface area is 100 m²/g or less, particles containing the Fe₁₆N₂ phase are resistant to aggregation, a uniform dispersion can be obtained easily, and a smooth surface can easily be obtained.

Synthesis of α-Fe

A process for producing particles containing the Fe₁₆N₂ phase is now explained. The Fe₁₆N₂ phase can be obtained by nitriding α-Fe. In order to obtain α-Fe, there is a method in which an iron-based oxide or hydroxide (e.g. hematite, magnetite, goethite) is reduced in the gas phase, and a method in which synthesis is carried out in the liquid phase. The method involving reduction in the gas phase is first explained. The average particle size of the iron-based oxide or hydroxide is not particularly limited, but it is preferable for it to normally be on the order of 5 to 100 nm. When the particle size is 5 nm or less, sintering between particles during a reduction treatment is suppressed, and when the particle size is 100 nm or less, the reduction treatment proceeds uniformly, and it is easy to control the particle size and the magnetic properties.

It is therefore preferable to cover the iron-based oxide or hydroxide by deposition with a compound containing a rare earth element or at least one type of element selected from boron, silicon, aluminum, phosphorus, etc., thus preventing sintering. Deposition of a rare earth element may be carried out by dispersing a starting material in an aqueous solution of an alkali or an acid, dissolving a salt of a rare earth element therein, and precipitating a hydroxide or a hydrate containing the rare earth element on the starting powder by a neutralization reaction, etc. When a compound containing at least one element selected from boron, silicon, aluminum, phosphorus, etc. is deposited, these compounds are dissolved in a solution in which a starting powder is immersed so as to effect adsorption or deposition, or deposition is carried out by precipitation.

A hydroxide or a hydrate may be deposited at the same time as or alternating with a rare earth element and at least one element selected from boron, silicon, aluminum, phosphorus, etc. In order to carry out such a deposition treatment efficiently, it is also preferable to add an additive such as a reducing agent, a pH buffer agent, or a particle size control agent.

Subsequently, the hydroxide or hydrate covered with the compound is heated in a flow of reducing gas. The reducing gas may be hydrogen gas or carbon monoxide gas. It is preferable to use hydrogen from the viewpoint of environmental suitability since it is converted into H₂O after the treatment.

The reduction temperature is preferably 250° C. to 600° C., and more preferably 300° C. to 500° C. The reduction reaction proceeds sufficiently in this temperature range, and sintering of particles can be prevented.

As a method for preventing particles from sintering during gas-phase reduction, a method in which α-Fe is synthesized in the liquid phase is preferably used. As processes for producing iron nanoparticles (iron particles having a nano-order size), there are known, when classified by precipitation technique, an alcohol reduction method employing a primary alcohol, a secondary alcohol, or a tertiary alcohol, a polyol reduction method employing a polyhydric alcohol such as a dihydric or trihydric alcohol, a thermal decomposition method, an ultrasonic decomposition method, and a strong reducing agent reduction method. Furthermore, with regard to the above-mentioned production process, when classified by reaction system, a method in the presence of a polymer, a high boiling point solvent method, a normal micelle method, a reverse micelle method, etc. are known.

The reverse micelle method, which can easily give a monodisperse dispersion due to easy control of the particle size, and is preferably used in the present invention, is now explained.

Reverse Micelle Synthetic Method for Iron Nanoparticles

A process for producing iron nanoparticles is explained below.

Iron nanoparticles may be produced by a reduction step in which a reverse micelle solution (I) containing at least one metal compound and a reverse micelle solution (II) containing a reducing agent are mixed and the mixture is subjected to a reduction treatment, and as necessary an aging step in which the mixture after the reduction treatment is subjected to an aging treatment. Iron nanoparticles are produced by such a production process. Each of the steps are explained below.

Reduction Step

First, the reverse micelle solution (I) in which a water-insoluble organic solvent containing a surfactant and an aqueous solution containing at least one type of metal compound are mixed is prepared. The reverse micelle solution (I) contains an iron salt used for the formation of iron nanoparticles.

As the surfactant, a lipid-soluble surfactant is used. Specific examples thereof include a sulfonic acid type (e.g. Aerosol OT (Wako Pure Chemical Industries, Ltd.)), a quaternary ammonium salt type (e.g. cetyltrimethylammonium bromide), and an ether type (e.g. pentaethylene glycol dodecyl ether).

As the water-insoluble organic solvent, which dissolves the surfactant, an alkane and an ether are preferable. The alkane is preferably an alkane having 7 to 12 carbons. Specific examples thereof include heptane, octane, nonane, decane, undecane, and dodecane. Preferred examples of the ether include diethyl ether, dipropyl ether, and dibutyl ether.

The amount of surfactant added to the water-insoluble organic solvent is preferably 20 to 200 g/L.

Examples of the metal compound contained in the aqueous solution of the metal compound include a hydracid of a metal complex having as a ligand a nitrate, sulfate, hydrochloride, acetate, or chloride ion, a potassium salt of a metal complex having a chloride ion as a ligand, a sodium salt of a metal complex having a chloride ion as a ligand, and an ammonium salt of a metal complex having an oxalate ion as a ligand, and the production process of the present invention may freely select these compounds and employ them.

The concentration of the metal compound in each of the aqueous solutions of the metal compounds is preferably 0.1 to 2,000 μmol/mL, and more preferably 1 to 500 μmol/mL.

In order for the particles thus obtained to have a uniform composition, it is preferable to add a chelating agent to the aqueous solution of the metal compound. Specifically, it is preferable to use as the chelating agent DHEG (dihydroxyethyl glycine), IDA (iminodiacetic acid), NTP (nitrilotripropionic acid), HIDA (dihydroxyethyliminodiacetic acid), EDDP (ethylenediamine dipropionic acid dihydrochloride), BAPTA (tetrapotassium bis(aminophenyl)ethylene glycol tetraacetate hydrate), etc. The chelate stability constant (log K) is preferably 10 or less.

The amount of chelating agent added is preferably 0.1 to 10 mol per mol of the metal compound, and more preferably 0.3 to 3 mol.

Subsequently, the reverse micelle solution (II) containing a reducing agent is prepared. The reverse micelle solution (II) may be prepared by mixing a water-insoluble organic solvent containing a surfactant and an aqueous solution of a reducing agent. When two or more types of reducing agents are used, they may be mixed together to give a reverse micelle solution (II), but taking into consideration the stability of the solutions, operability, etc., they may preferably be separately mixed with water-insoluble organic solvents to give separate reverse micelle solutions ((II′), (II″), etc.), and these solutions may appropriately be mixed and used.

The aqueous solution of the reducing agent comprises, for example, an alcohol, a polyhydric alcohol, H₂, HCHO, S₂O₆²⁻, H₂PO₂⁻, BH₄⁻, N₂H₅⁺, H₂PO₃⁻, etc. and water, and these reducing agents may be used singly or in a combination of two or more types.

The amount of reducing agent in the aqueous solution is preferably 3 to 50 mol per mol of metal salt.

With regard to the surfactant and the water-insoluble organic solvent used in the reverse micelle solution (II), those cited for the reverse micelle solution (I) can be used.

The ratio by weight (water/surfactant) of water to surfactant contained in each of the reverse micelle solutions (I) and (II) is preferably 20 or less. When the ratio by weight is 20 or less, precipitation is suppressed, and uniform particles can be obtained. The ratio by weight is more preferably 15 or less, and yet more preferably 0.5 to 10.

The ratios by weight of water to surfactant of the reverse micelle solutions (I) and (II) may be identical to or different from each other, but in order to give a uniform system the ratios by weight are preferably identical to each other.

The reverse micelle solutions (I) and (II) thus prepared are mixed. A mixing method is not particularly limited, but taking into consideration uniformity of reduction it is preferable to add the reverse micelle solution (II) to the reverse micelle solution (I) while stirring. After completion of the mixing, a reduction reaction is effected, and the temperature during the reaction is a constant temperature in the range of −5° C. to 30° C. When the reduction temperature is −5° C. or higher, the aqueous phase does not freeze and the reduction reaction can be carried out uniformly, and when it is 30° C. or less, aggregation or precipitation is suppressed, and the system can be stabilized. The reduction temperature is preferably 0° C. to 25° C., and more preferably 5° C. to 25° C.

The ‘constant temperature’ referred to above means that when a set temperature is T (° C.) the temperature is in the range of T±3° C. Even in such a case, the upper limit and the lower limit of said T are within the above-mentioned range for the reduction temperature (−5° C. to 30° C.).

It is necessary to set a time for the reduction reaction as appropriate depending on the amounts of the reverse micelle solutions (I) and (II), etc., but it is preferably 1 to 30 minutes, and more preferably 5 to 20 minutes.

Since the reduction reaction greatly affects the monodispersity of the iron particle size distribution, it is preferably carried out while stirring at as high a speed as possible (e.g. about 3,000 rpm or greater).

A preferred stirring device is a stirring device having high shear force and, more particularly, a stirring device having a structure in which a stirring vane is basically a turbine type or a paddle type and, furthermore, a structure in which a sharp blade is mounted at the end of the vane or at a position bordering the vane, and the vane is rotated by means of a motor. Specifically, a Dissolver (Primix Corporation), an Omnimixer (Yamato Scientific Co., Ltd.), a Homogenizer (SMT Co., Ltd.), etc. are useful. By using these devices, it is possible to synthesize monodisperse nanoparticles as a stable dispersion.

After the reaction between the reverse micelle solutions (I) and (II), it is preferable to add, per mol of the iron nanoparticles that are to be produced, 0.001 to 10 mol of at least one type of dispersant having 1 to 3 amino groups or carboxy groups. When the amount of dispersant added is 0.001 to 10 mol, the monodispersity of the iron nanoparticles can be improved, and aggregation is prevented.

As the dispersant, an organic compound having a group that adsorbs on the surface of iron nanoparticles is preferable. Specific examples thereof include those having 1 to 3 amino groups, carboxy groups, sulfonic acid groups, or sulfinic acid groups, and they may be used singly or in combination.

These compounds have the structural formulae R—NH₂, H₂N—R—NH₂, H₂N—R(NH₂)—NH₂, R—COOH, HOCO—R—COOH, HOCO—R(COOH)—COOH, R—SO₃H, HOSO₂—R—SO₃H, HOSO₂—R(SO₃H)—SO₃H, R—SO₂H, HOSO—R—SO₂H, or HOSO—R(SO₂H)—SO₂H, in which R is a straight-chain, branched, or cyclic saturated or unsaturated hydrocarbon residue.

A particularly preferred compound as the dispersant is oleic acid. Oleic acid is a well-known surfactant for stabilizing a colloid, and is used for protecting iron nanoparticles. The relatively long chain of oleic acid gives an important steric hindrance that counteracts the strong magnetic interaction between particles (oleic acid has a chain of 18 carbons, a length of on the order of 2 nm (20 Å), and has one double bond). Oleic acid is preferable since it is an inexpensive natural resource easily available from, for example, olive oil. Oleylamine, which is derived from oleic acid, is also a useful dispersant in the same way as oleic acid.

In addition, a similar long-chain carboxylic acid such as erucic acid or linoleic acid can also be used in the same way as oleic acid (e.g. long-chain organic acids having 8 to 22 carbon atoms may be used singly or in combination).

The timing of addition of the dispersant is not particularly limited, but it is preferably from immediately after the reduction reaction to the start of an aging step described below. By adding such a dispersant, monodisperse iron nanoparticles free from aggregation can be obtained.

Aging Step

The production process of the present invention further comprises, after completion of the reduction reaction, an aging step in which the temperature of the reaction solution is increased to an aging temperature.

The aging temperature is preferably a constant temperature between 30° C. to 90° C., and it is desirable that the aging temperature is higher than the temperature of the reduction reaction. The aging time is preferably 5 to 180 minutes. When the aging temperature and the aging time are in the above-mentioned ranges, aggregation and precipitation are suppressed, the reaction can be completed, and the composition can be made uniform. More preferred aging temperature and aging time are 40° C. to 80° C. and 10 to 150 minutes, and yet more preferred aging temperature and aging time are 40° C. to 70° C. and 20 to 120 minutes.

The ‘constant temperature’ referred to here has the same meaning as in the case of the temperature of the reduction reaction (however, in this case ‘reduction temperature’ is ‘aging temperature’) and, in particular, within the above-mentioned range for the aging temperature (30° C. to 90° C.), the aging temperature is preferably higher than the temperature of the reduction reaction by 5° C. or greater, and more preferably by 10° C. or greater. By making said temperature higher by 5° C. or greater, a composition as prescribed can be obtained.

In the above-mentioned aging step, iron nanoparticles having a desired particle size can be prepared by appropriately adjusting a stirring speed at a given aging temperature.

It is preferable to provide washing and dispersion steps after carrying out the aging step such that the solution after aging is washed with a mixed solution of water and a primary alcohol, then subjected to a precipitation treatment with a primary alcohol so as to form a precipitate, and this precipitate is dispersed in an organic solvent. By providing such washing and dispersion steps, impurities are removed, and coating properties during the formation of a magnetic layer of the magnetic recording medium can be improved.

The above-mentioned washing and dispersion steps may be carried out at least once each, and preferably at least two times each.

The primary alcohol used in washing is not particularly limited, but methanol, ethanol, etc. are preferable. The mixing ratio by volume of water and the primary alcohol (water/primary alcohol) is preferably in the range of 10/1 to 2/1, and more preferably in the range of 5/1 to 3/1. When the mixing ratio by volume of water and the primary alcohol is in the above-mentioned range, surfactant can easily be removed, and aggregation is suppressed.

When iron is reductively precipitated or thermally precipitated, the presence of a protecting colloid enables nanoparticles to be prepared stably. For thermal precipitation, a method is known in which iron carbonyl is thermally decomposed to give iron. As the protecting colloid, a polymer or a surfactant is preferably used. Examples of the polymer include polyvinyl alcohol (PVA), poly(N-vinyl-2-pyrrolidone) (PVP), and gelatin. Among them, PVP is particularly preferable. The molecular weight is preferably 20,000 to 60,000, and more preferably 30,000 to 50,000. The amount of polymer is preferably 0.1 to 10 times the weight of hard magnetic nanoparticles produced, and more preferably 0.1 to 5 times.

The surfactant preferably used as the protecting colloid preferably contains an ‘organic stabilizer’, which is a long-chain organic compound represented by the Formula R—X. In the above-mentioned formula, R is a ‘tail group’, which is a straight-chain or branched hydrocarbon or fluorocarbon chain, and normally contains 8 to 22 carbon atoms. X in the above formula is a ‘head group’, which is a moiety (X) providing a specific chemical bond to the nanoparticle surface, and is preferably any one of sulfinate (—SOOH), sulfonate (—SO₂OH), phosphinate (-POOH), phosphonate (—OPO(OH)₂), carboxylate, and thiol.

The organic stabilizer is preferably any one of a sulfonic acid (R—SO₂OH), a sulfinic acid (R—SOOH), a phosphinic acid (R₂POOH), a phosphonic acid (R—OPO (OH)₂), a carboxylic acid (R—COOH), and a thiol (R—SH). Among them, oleic acid is particularly preferable.

Oleic acid is a well-known surfactant for stabilizing a colloid, and is suitable for protecting iron-based nanoparticles. Oleic acid has an 18 carbon chain, and its length is about 20 Å (about 2 nm). Oleic acid is not a saturated fatty acid and has one double bond. The relatively long chain of oleic acid gives an important steric hindrance that counteracts the strong magnetic interaction between particles. A similar long-chain carboxylic acid such as erucic acid or linoleic acid has also been used in the same way as oleic acid (e.g. long-chain organic acids having 8 to 22 carbon atoms may be used singly or in combination), but oleic acid is particularly preferable since it is an inexpensive natural resource easily available from, for example, olive oil.

A combination of a phosphine and the organic stabilizer (triorganophosphine/ acid, etc.) can provide excellent controlability for the growth and stabilization of particles. It is also possible to use didecyl ether and didodecyl ether, but phenyl ether or n-octyl ether is suitably used as a solvent due to low cost and high boiling point.

The reaction is preferably carried out at a temperature in the range of 80° C. to 360° C. depending on the nanoparticles required and the boiling point of the solvent, and a temperature between 80° C. and 240° C. is preferable. When the temperature is in the above-mentioned range, particles grow sufficiently, controlability of the growth of particles is excellent, and the formation of by-products can be suppressed.

In order to increase the particle size, a seed crystal method is preferably used. In this case, since there is a possibility that seed crystal iron particles might be oxidized, the particles are preferably hydrogenated in advance.

It is preferable to remove a salt from the solution after synthesizing the iron nanoparticles since the dispersion stability of the nanoparticles is improved. For desalting, there is a method in which excess alcohol is added so as to cause a light degree of aggregation, and after natural sedimentation or centrifugal sedimentation the salt is removed together with the supernatant, but in such a method aggregation easily occurs, and it is therefore preferable to employ an ultrafiltration method.

Nitriding

Prior to nitriding, when there is a possibility of oxidation of iron nanoparticles, they may be subjected to a reduction treatment in a flow of gas such as hydrogen or a mixed gas of hydrogen and an inert gas (H₂, Ar, He, etc.). The temperature is preferably 200° C. to 300° C., and more preferably 250° C. to 300° C. When it is in the above-mentioned range, fusion of particles does not occur, and the reduction can be carried out sufficiently.

Heating iron nanoparticles in a flow of a nitrogen-containing gas enables the Fe₁₆N₂ phase to be obtained.

As a nitriding gas, nitrogen gas, a nitrogen+hydrogen gas mixture, ammonia gas, etc. may be used, and the use of ammonia gas is convenient.

Nitriding in an NH₃ atmosphere is preferably carried out in a flow of ammonia (NH₃) or in a flow of a mixed gas containing ammonia gas (e.g. a mixed gas containing ammonia gas and at least one of argon, hydrogen, and nitrogen) at a relatively low temperature in the range of 100° C. to 250° C. When the nitriding temperature is in the above-mentioned range, a sufficient amount of the Fe₁₆N₂ phase can be obtained, and formation of the Fe₁₆N₂ phase progresses sufficiently quickly. It is preferable for these gases to be highly pure (5N or higher) or to contain oxygen at a few ppm or less.

It is industrially preferable for it to be carried out at a temperature in the range of 100° C. to 250° C. for 0.5 to 48 hours, and the treatment time is more preferably 0.5 to 24 hours, although it depends on the particle size.

When carrying out such nitriding, it is desirable that the conditions for nitriding are selected so that the content of nitrogen relative to iron in the magnetic powder obtained is 1.0 to 20 atom %. When the nitrogen content is 1.0 atom % or greater, the amount of Fe₁₆N₂ formed is sufficient, and there is a sufficient effect in improving the coercivity. When the nitrogen content is 20 atom % or less, the formation of an Fe₄N or Fe₃N phase is suppressed, a sufficient coercivity can be obtained, and the amount of saturation magnetization is appropriate.

Oxide Coating

In order to form an oxide coating, an oxide coating having the above-mentioned thickness can be formed by carrying out a treatment under an atmosphere of an inert gas (N₂, Ar, He, Ne, etc.) having an oxygen concentration of 1% to 5% at a temperature of 0° C. to 100° C. for 1 to 10 hours.

In order to provide a covering of a rare earth element, a starting material is normally dispersed in an aqueous solution of an alkali or an acid, a salt of the rare earth element is dissolved therein, and a hydroxide or hydrate containing the rare earth element may be deposited so as to cover particles mainly containing Fe₁₆N₂ by a neutralization reaction, etc.

A compound formed from silicon or aluminum and, furthermore, an element such as boron or phosphorus as necessary is dissolved, particles mainly containing Fe₁₆N₂ are immersed therein, and silicon or aluminum may be deposited so as to cover the particles mainly containing Fe₁₆N₂. In order to efficiently carry out such a deposition process, an additive such as a reducing agent, a pH buffer agent, or a particle size control agent may be added.

In this deposition process, a rare earth element and silicon, aluminum, etc. may be deposited at the same time or alternately.

Binder

In the present invention, a conventionally known thermoplastic resin, thermosetting resin, reactive resin or a mixture thereof is used as a binder of the magnetic layer.

The thermoplastic resin preferably has a glass transition temperature of −100° C. to 150° C., a number-average molecular weight of 1,000 to 200,000, and more preferably 10,000 to 100,000, and a degree of polymerization of 50 to 1,000.

Examples thereof include polymers and copolymers containing as a repeating unit vinyl chloride, vinyl acetate, vinyl alcohol, maleic acid, acrylic acid, an acrylate ester, vinylidene chloride, acrylonitrile, methacrylic acid, a methacrylate ester, styrene, butadiene, ethylene, vinyl butyral, vinyl acetal and vinyl ether; polyurethane resins; and various types of rubber resins.

Examples of the thermosetting resin and the reactive resin include phenol resins, epoxy resins, curable type polyurethane resins, urea resins, melamine resins, alkyd resins, reactive acrylic resins, formaldehyde resins, silicone resins, epoxy-polyamide resins, mixtures of a polyester resin and an isocyanate prepolymer, mixtures of a polyester polyol and a polyisocyanate, and mixtures of a polyurethane and a polyisocyanate.

Details of these resins are described in the ‘Purasuchikku Binran’ (Plastic Handbook) published by Asakura Shoten. It is also possible to use a known electron beam curable type resin in the non-magnetic layer (lower layer) or the magnetic layer (upper layer). Examples of the resin and a production method therefor are disclosed in detail in JP-A-62-256219. The above-mentioned resins can be used singly or in combination. Combinations of a polyurethane resin with at least one selected from a vinyl chloride resin, a vinyl chloride-vinyl acetate resin, a vinyl chloride-vinyl acetate-vinyl alcohol resin, a vinyl chloride-vinyl acetate-maleic anhydride copolymer, and nitrocellulose, and combinations thereof with a polyisocyanate are preferred.

Specific examples of the binder include VAGH, VYHH, VMCH, VAGF, VAGD, VROH, VYES, VYNC, VMCC, XYHL, XYSG, PKHH, PKHJ, PKHC and PKFE (manufactured by Union Carbide Corporation), MPR-TA, MPR-TA5, MPR-TAL, MPR-TSN, MPR-TMF, MPR-TS, and MPR-TM (manufactured by Nisshin Chemical Industry Co., Ltd.), 1000W, DX80, DX81, DX82, and DX83 (manufactured by Denki Kagaku Kogyo Kabushiki Kaisha), MR-110, MR-100, and 400X-110A (manufactured by Nippon Zeon Corporation), Nippollan N2301, N2302, and N2304 (manufactured by Nippon Polyurethane Industry Co., Ltd.), Pandex T-5105, T-R3080, and T-5201, Burnock D-400 and D-210-80, and Crisvon 6109 and 7209 (manufactured by Dainippon Ink and Chemicals, Incorporated), Vylon UR8200, UR8300, RV530, and RV280 (manufactured by Toyobo Co., Ltd.), Daiferamine 4020, 5020, 5100, 5300, 9020, 9022, and 7020 (manufactured by Dainichiseika Color & Chemicals Mfg. Co., Ltd.), MX5004 (manufactured by Mitsubishi Chemical Corp.), Sanprene SP-150 (manufactured by Sanyo Chemical Industries, Ltd.), and Saran F310 and F210 (manufactured by Asahi Kasei Corporation).

As the binder that can be used in the magnetic layer, among the above-mentioned binders, a vinyl chloride-based binder or a polyurethane-based binder is preferable, and a polyurethane containing a polar group and containing 3.5 mmol/g to 7 mmol/g of aromatic rings in the framework is particularly preferable.

Preferred examples of the polyurethane-based binder include polyester urethane, polyether urethane, polycarbonate urethane, polyether ester urethane, and acrylic polyurethane. The above-mentioned polyurethane-based binders are preferable since they have high affinity for the above-mentioned lubricant and the amount of surface lubricant can be controlled so as to be in an optimum range.

The polar group that the binder may have is preferably a sulfonate, a sulfamate, a sulfobetaine, a phosphate, a phosphonate, etc. The amount of polar group is preferably 1×10⁻⁵ eq/g to 2×10⁻⁴ eq/g.

The amount of binder, including curing agent, in the magnetic layer is preferably 10 to 25 parts by weight relative to 100 parts by weight of the ferromagnetic powder, the amount of binder in the non-magnetic lower layer is preferably 25 to 40 parts by weight relative to 100 parts by weight of the non-magnetic powder, and with regard to the amounts of binder in the magnetic layer and the non-magnetic lower layer, it is preferable to add a larger amount of binder to the lower layer.

In particular, the binder for the non-magnetic lower layer preferably has a framework containing a strongly polar group such as SO₃Na and a large number of aromatic groups. This enables the affinity between the lubricant and the non-magnetic lower layer binder to be increased, and allows a large amount of lubricant to be present in the non-magnetic lower layer in a stable manner.

When the affinity between the lubricant and the binder is appropriate, the binder and the lubricant are not completely miscible at the molecular level, and the lubricant can move to the upper layer, which is preferable.

Abrasive

The magnetic layer of the magnetic recording medium of the present invention preferably contains an abrasive.

An inorganic non-magnetic powder can be used as the abrasive. Examples of the inorganic non-magnetic powder include inorganic compounds such as a metal oxide, a metal carbonate, a metal sulfate, a metal nitride, a metal carbide, and a metal sulfide. As the inorganic compound, α-alumina with an α-component proportion of 90% to 100%, β-alumina, γ-alumina, silicon carbide, chromium oxide, cerium oxide, α-iron oxide (colcothar), corundum, silicon nitride, titanium carbide, titanium oxide, silicon dioxide, tin oxide, magnesium oxide, tungsten oxide, zirconium oxide, boron nitride, zinc oxide, barium sulfate, molybdenum disulfide, etc. can be used singly or in combination. Particularly preferred are α-alumina, colcothar, and chromium oxide.

The abrasive that can be used in the present invention is used by varying the type, amount, particle size, combination, shape, etc. so that the ratio H₁₅/H₁₀, which denotes the protrusion height distribution of the abrasive present on the surface of the magnetic layer, is in the above-mentioned range. When only one type of abrasive is used, the average particle size of the abrasive used in the present invention is preferably 0.05 to 0.4 μm, and more preferably 0.1 to 0.3 μm. It is preferable that particles with a particle size larger than the average particle size by 0.1 μm or more are present at a proportion of 1 to 40%, more preferably 5 to 30%, and most preferably 10 to 20%. Although the particle size of the abrasive itself affects the particle size of abrasive particles that are actually present on the surface of the magnetic layer, they are not equal to each other. The particle size of the abrasive particles present on the surface of the magnetic layer varies according to the dispersion conditions, etc. for the abrasive. Furthermore, some particles come out easily to the surface of the magnetic layer during coating and drying steps whereas it is difficult for others to come out to the surface.

Two or more abrasives having different average particle sizes may be used in combination. In this case, taking the weighted average value as the average particle size, which depends on the actual proportions used of the two or more abrasives, the particles with the average particle size and the particles with a particle size 0.1 μm or more greater than the average particle size can be set so as to be within the above-mentioned ranges.

Changing the dispersion conditions for the two abrasives can also control the particle size. For example, abrasive A is dispersed with a binder and a solvent in advance. This dispersion and abrasive B as a powder are added to a kneaded ferromagnetic metal powder that has been kneaded separately with a binder and a solvent, and the mixture is dispersed. In this way, the dispersion conditions for the abrasive A and the abrasive B can be varied. That is, the abrasive A is dispersed more strongly than the abrasive B. The tap density of the abrasive powder is preferably 0.05 to 2 g/mL, and more preferably 0.2 to 1.5 g/mL.

The water content of the abrasive powder is preferably 0.05 to 5 wt %, and more preferably 0.2 to 3 wt %. The specific surface area of the abrasive is preferably 1 to 100 m²/g, and more preferably 5 to 50 m²/g. Its oil absorption determined using DBP (dibutyl phthalate) is preferably 5 to 100 mL/100 g, and more preferably 10 to 80 mL/100 g. The specific gravity is preferably 1 to 12, and more preferably 3 to 6. The shape of the abrasive may be any one of acicular, spherical, polyhedral, and tabular. The surface of the abrasive may be coated at least partially with a compound which is different from the main component of the abrasive. Examples of the compound include Al₂O₃, SiO₂, TiO₂, ZrO₂, SnO₂, Sb₂O₃, and ZnO. In particular, the use of A1₂0₃, SiO₂, TiO₂ or ZrO₂ gives good dispersibility. These compounds may be used singly or in combination.

Specific examples of the abrasive that can be used in the magnetic layer of the present invention include Nanotite (manufactured by Showa Denko K.K.), Hit 100, Hit 82, Hit 80, Hit 70, Hit 60A, Hit 55, AKP-20, AKP-30, AKP-50, and ZA-G1 (manufactured by Sumitomo Chemical Co., Ltd.), ERC-DBM, HP-DBM, HPF-DBM, HPFX-DBM, HPS-DBM, and HPSX-DBM (manufactured by Reynolds Corp.), WA8000 and WA10000 (manufactured by Fujimi Incorporated), UB20, UB40B, and Mecanox UA (manufactured by C. Uyemura & Co., Ltd.), UA2055, UA5155, and UA5305 (manufactured by Showa Keikinzoku K.K.), G-5, Kromex M, Kromex S1, Kromex U2, Kromex U1, Kromex X10, and Kromex KX10 (manufactured by Nippon Chemical Industry Co., Ltd.), ND803, ND802, and ND801 (manufactured by Nippon Denko Co., Ltd.), F-1, F-2, and UF-500 (manufactured by Tosoh Corporation), DPN-250, DPN-250BX, DPN-245, DPN-270BX, TF-100, TF-120, TF-140, DPN-550BX, and TF-180 (manufactured by Toda Kogyo Corp.), A-3 and B-3 (manufactured by Showa Mining Co., Ltd.), beta SiC and UF (manufactured by Central Glass Co., Ltd.), β-Random Standard and β-Random Ultrafine (manufactured by Ibiden Co., Ltd.), JR401, MT-100S, MT-100T, MT-150W, MT-500B, MT-600B, MT-100F, and MT-500HD (manufactured by Tayca Corporation), TY-50, TTO-51 B, TTO-55A, TTO-55B, TTO-55C, TTO-55S, TTO-55D, SN-100, E270, and E271 (manufactured by Ishihara Sangyo Kaisha Ltd.), STT-4D, STT-30D, STT-30, STT-65C, and Y-LOP, and calcined products thereof (manufactured by Titan Kogyo Kabushiki Kaisha), FINEX-25, BF-1, BF-10, BF-20, and ST-M (manufactured by Sakai Chemical Industry Co., Ltd.), HZn and HZr3M (manufactured by Hokkai Kagaku), DEFIC-Y and DEFIC-R (manufactured by Dowa Mining Co., Ltd.), AS2BM and TiO₂P25 (manufactured by Nippon Aerosil Co., Ltd.), and 100A and 500A (manufactured by Ube Industries, Ltd.).

Additives

The magnetic layer of the magnetic recording medium of the present invention can comprise an additive as necessary. Examples of the additive include a dispersant/dispersion adjuvant, a fungicide, an antistatic agent, an antioxidant, a solvent, and carbon black. Furthermore, a lubricant other than the above-mentioned carbonic acid ester may be used in combination as an additive.

Examples of these additives include tungsten disulfide, graphite, graphite fluoride, a silicone oil, a polar group-containing silicone, a fatty acid-modified silicone, a fluorine-containing silicone, a fluorine-containing alcohol, a fluorine-containing ester, a polyolefin, a polyglycol, a polyphenyl ether; aromatic ring-containing organic phosphonic acids such as phenylphosphonic acid, benzylphosphonic acid, phenethylphosphonic acid, α-methylbenzylphosphonic acid, 1-methyl-1-phenethylphosphonic acid, diphenylmethylphosphonic acid, biphenylphosphonic acid, benzylphenylphosphonic acid, α-cumylphosphonic acid, tolylphosphonic acid, xylylphosphonic acid, ethylphenylphosphonic acid, cumenylphosphonic acid, propylphenylphosphonic acid, butylphenylphosphonic acid, heptylphenylphosphonic acid, octylphenylphosphonic acid, and nonylphenylphosphonic acid, and alkali metal salts thereof; alkylphosphonic acids such as octylphosphonic acid, 2-ethylhexylphosphonic acid, isooctylphosphonic acid, isononylphosphonic acid, isodecylphosphonic acid, isoundecylphosphonic acid, isododecylphosphonic acid, isohexadecylphosphonic acid, isooctadecylphosphonic acid, and isoeicosylphosphonic acid, and alkali metal salts thereof; aromatic phosphates such as phenyl phosphate, benzyl phosphate, phenethyl phosphate, α-methylbenzyl phosphate, 1-methyl-1-phenethyl phosphate, diphenylmethyl phosphate, biphenyl phosphate, benzylphenyl phosphate, α-cumyl phosphate, tolyl phosphate, xylyl phosphate, ethylphenyl phosphate, cumenyl phosphate, propylphenyl phosphate, butylphenyl phosphate, heptylphenyl phosphate, octylphenyl phosphate, and nonylphenyl phosphate, and alkali metal salts thereof; alkyl phosphates such as octyl phosphate, 2-ethylhexyl phosphate, isooctyl phosphate, isononyl phosphate, isodecyl phosphate, isoundecyl phosphate, isododecyl phosphate, isohexadecyl phosphate, isooctadecyl phosphate, and isoeicosyl phosphate, and alkali metal salts thereof; alkyl sulfonates and alkali metal salts thereof; fluorine-containing alkyl sulfates and alkali metal salts thereof; monobasic fatty acids that have 10 to 24 carbons, may contain an unsaturated bond, and may be branched, such as lauric acid, myristic acid, palmitic acid, stearic acid, behenic acid, oleic acid, linoleic acid, linolenic acid, elaidic acid, and erucic acid, and metal salts thereof; mono-fatty acid esters, di-fatty acid esters, and poly-fatty acid esters such as butyl stearate, octyl stearate, amyl stearate, isooctyl stearate, octyl myristate, butyl laurate, butoxyethyl stearate, anhydrosorbitan monostearate, anhydrosorbitan distearate, and anhydrosorbitan tristearate that are formed from a monobasic fatty acid that has 10 to 24 carbons, may contain an unsaturated bond, and may be branched, and any one of a mono- to hexa-hydric alcohol that has 2 to 22 carbons, may contain an unsaturated bond, and may be branched, an alkoxy alcohol that has 12 to 22 carbons, may have an unsaturated bond, and may be branched, and a mono alkyl ether of an alkylene oxide polymer; fatty acid amides having 2 to 22 carbons; aliphatic amines having 8 to 22 carbons; etc. Other than the above-mentioned hydrocarbon groups, those having an alkyl, aryl, or aralkyl group that is substituted with a group other than a hydrocarbon group, such as a nitro group, F, Cl, Br, or a halogen-containing hydrocarbon such as CF₃, CCl₃, or CBr₃ can also be used.

Furthermore, there are a nonionic surfactant such as an alkylene oxide type, a glycerol type, a glycidol type, or an alkylphenol-ethylene oxide adduct; a cationic surfactant such as a cyclic amine, an ester amide, a quaternary ammonium salt, a hydantoin derivative, a heterocyclic compound, a phosphonium salt, or a sulfonium salt; an anionic surfactant containing an acidic group such as a carboxylic acid, a sulfonic acid, or a sulfate ester group; and an amphoteric surfactant such as an amino acid, an aminosulfonic acid, a sulfate ester or a phosphate ester of an amino alcohol, or an alkylbetaine. Details of these surfactants are described in ‘Kaimenkasseizai Binran’ (Surfactant Handbook) (published by Sangyo Tosho Publishing).

The additives such as these dispersants and the lubricants used in combination need not always be pure and may contain, in addition to the main component, an impurity such as an isomer, an unreacted material, a by-product, a decomposition product, or an oxide. However, the impurity content is preferably 30 wt % or less, and more preferably 10 wt % or less.

Specific examples of these additives include NAA-102, hardened castor oil fatty acid, NAA-42, Cation SA, Nymeen L-201, Nonion E-208, Anon BF, and Anon LG (produced by Nippon Oil & Fats Co., Ltd.), FAL-205 and FAL-123 (produced by Takemoto Oil & Fat Co., Ltd), Enujelv OL (produced by New Japan Chemical Co., Ltd.), TA-3 (produced by Shin-Etsu Chemical Industry Co., Ltd.), Armide P (produced by Lion Armour), Duomin TDO (produced by Lion Corporation), BA-41G (produced by The Nisshin Oil Mills, Ltd.), and Profan 2012E, Newpol PE 61, and lonet MS-400 (produced by Sanyo Chemical Industries, Ltd.).

An organic solvent used for the magnetic layer of the magnetic recording medium of the present invention can be a known organic solvent. As the organic solvent, a ketone such as acetone, methyl ethyl ketone, methyl isobutyl ketone, diisobutyl ketone, cyclohexanone, or isophorone, an alcohol such as methanol, ethanol, propanol, butanol, isobutyl alcohol, isopropyl alcohol, or methylcyclohexanol, an ester such as methyl acetate, butyl acetate, isobutyl acetate, isopropyl acetate, ethyl lactate, or glycol acetate, a glycol ether such as glycol dimethyl ether, glycol monoethyl ether, or dioxane, an aromatic hydrocarbon such as benzene, toluene, xylene, or cresol, a chlorohydrocarbon such as methylene chloride, ethylene chloride, carbon tetrachloride, chloroform, ethylene chlorohydrin, chlorobenzene, or dichlorobenzene, N,N-dimethylformamide, hexane, tetrahydrofuran, etc. can be used at any ratio.

These organic solvents do not always need to be 100% pure, and may contain an impurity such as an isomer, an unreacted compound, a by-product, a decomposition product, an oxide, or moisture in addition to the main component. The content of these impurities is preferably 30% or less, and more preferably 10% or less. The organic solvent used in the present invention is preferably the same type for both the magnetic layer and the non-magnetic layer. However, the amount added may be varied. The coating stability is improved by using a high surface tension solvent (cyclohexanone, dioxane, etc.) for the non-magnetic layer; more specifically, it is important that the arithmetic mean value of the surface tension of the upper layer solvent composition is not less than that for the surface tension of the non-magnetic layer solvent composition. In order to improve the dispersibility, it is preferable for the polarity to be somewhat strong, and the solvent composition preferably contains 50% or more of a solvent having a permittivity of 15 or higher. The solubility parameter is preferably 8 to 11.

These dispersants and surfactants used in the magnetic layer of the magnetic recording medium of the present invention may be selected as necessary in terms of the type and amount according to the magnetic layer and the non-magnetic layer, which will be described later. For example, although these examples should not be construed as being limited thereto, the dispersant has the property of adsorbing or bonding via its polar group, and it is adsorbed on or bonds to the surface of mainly the ferromagnetic powder in the magnetic layer and the surface of mainly a non-magnetic powder in the non-magnetic layer, which will be described later, via the polar group; it is surmised that once an organophosphorus compound has been adsorbed on the surface of a metal, a metal compound, etc. it is difficult for it to desorb. In the present invention, the surface of the ferromagnetic powder or the surface of the non-magnetic powder is therefore covered with an alkyl group, an aromatic group, etc., the affinity of the ferromagnetic powder or the non-magnetic powder toward the binder resin component increases, and the dispersion stability of the ferromagnetic powder or the non-magnetic powder is also improved. Furthermore, it is though that, for example, by adjusting the amount of surfactant the coating stability is improved. All or a part of the additives used in the present invention may be added to a magnetic coating solution or a non-magnetic coating solution at any stage of its preparation. For example, the additives may be blended with a ferromagnetic powder prior to a kneading step, they may be added in a step of kneading a ferromagnetic powder, a binder, and a solvent, they may be added in a dispersing step, they may be added after dispersion, or they may be added immediately prior to coating.

The magnetic layer of the magnetic recording medium of the present invention can contain as necessary carbon black.

Types of carbon black that can be used include furnace black for rubber, thermal black for rubber, black for coloring, and acetylene black. The carbon black should have characteristics that have been optimized as follows according to a desired effect, and the effect can be increased by the use thereof in combination.

The specific surface area of the carbon black is preferably 100 to 500 m²/g, and more preferably 150 to 400 m²/g, and the DBP oil absorption is preferably 20 to 400 mL/100 g, and more preferably 30 to 200 mL/100 g. The particle size of the carbon black is preferably 5 to 80 nm, more preferably 10 to 50 nm, and yet more preferably 10 to 40 nm. The pH of the carbon black is preferably 2 to 10, the water content thereof is preferably 0.1% to 10%, and the tap density is preferably 0.1 to 1 g/mL.

Specific examples of the carbon black that can be used in the present invention include BLACKPEARLS 2000, 1300, 1000, 900, 800, 880 and 700, and VULCAN XC-72 (manufactured by Cabot Corporation), #3050B, #3150B, #3250B, #3750B, #3950B, #950, #650B, #970B, #850B, MA-600, MA-230, #4000, and #4010 (manufactured by Mitsubishi Chemical Corporation), CONDUCTEX SC, and RAVEN 8800, 8000, 7000, 5750, 5250, 3500, 2100, 2000, 1800, 1500, 1255, and 1250 (manufactured by Columbian Carbon Co.), Ketjen Black EC (manufactured by Akzo), and Ketjen Black EC (manufactured by Ketjen Black International Company Ltd.).

The carbon black may be subjected to any of a surface treatment with a dispersant, etc., grafting with a resin, or a partial surface graphitization. The carbon black may also be dispersed in a binder prior to addition to a coating solution. The carbon black that can be used in the present invention can be selected by referring to, for example, the ‘Kabon Burakku Handobukku’ (Carbon Black Handbook) (edited by the Carbon Black Association of Japan).

The carbon black may be used singly or in a combination of different types thereof. When the carbon black is used, it is preferably used in an amount of 0.1 to 30 wt % based on the weight of the magnetic substance. The carbon black has the functions of preventing static charging of the magnetic layer, reducing the coefficient of friction, imparting light-shielding properties, and improving the film strength. Such functions vary depending upon the type of carbon black. Accordingly, it is of course possible in the present invention to appropriately choose the type, the amount and the combination of carbon black for the magnetic layer according to the intended purpose on the basis of the above mentioned various properties such as the particle size, the oil absorption, the electrical conductivity, and the pH value, and it is better if they are optimized for the respective layers.

II. Non-Magnetic Layer

The non-magnetic layer (non-magnetic lower layer, lower coated layer) is now explained in detail.

The magnetic recording medium of the present invention may comprise, between the non-magnetic support and the magnetic layer, at least one non-magnetic layer comprising a non-magnetic powder dispersed in a binder.

The binder is preferably the same binder as that of the magnetic layer.

Non-Magnetic Powder

The non-magnetic powder used in the non-magnetic layer may be an inorganic material or an organic material. The non-magnetic layer may contain, together with the non-magnetic powder, carbon black as necessary.

The inorganic powder used in the lower coated layer is a non-magnetic powder, and may be selected from, for example, inorganic compounds such as a metal oxide, a metal carbonate, a metal sulfate, a metal nitride, a metal carbide, and a metal sulfide.

With regard to the inorganic compounds, for example, α-alumina with an α component proportion of at least 90%, β-alumina, γ-alumina, θ-alumina, silicon carbide, chromium oxide, cerium oxide, α-iron oxide, goethite, corundum, silicon nitride, titanium carbide, titanium oxide, silicon dioxide, tin oxide, magnesium oxide, tungsten oxide, zirconium oxide, boron nitride, zinc oxide, calcium carbonate, calcium sulfate, barium sulfate, molybdenum disulfide, etc. can be used singly or in combination. From the viewpoint of a narrow particle size distribution, the possibility of having many means for imparting functionality, etc., titanium dioxide, zinc oxide, iron oxide, and barium sulfate are particularly preferable, and titanium dioxide and α-iron oxide are more preferable.

The particle size of such a non-magnetic powder is preferably 0.005 to 2 μm, but it is also possible, as necessary, to combine non-magnetic powders having different particle sizes or widen the particle size distribution of a single non-magnetic powder, thus producing the same effect. The particle size of the non-magnetic powder is particularly preferably 0.01 to 0.2 μm. In particular, when the non-magnetic powder is a granular metal oxide, the average particle size is preferably 0.08 μm or less, and when it is an acicular metal oxide, the major axis length is preferably 0.3 μm or less. The tap density is preferably 0.05 to 2 g/mL, and more preferably 0.2 to 1.5 g/mL. The water content of the non-magnetic powder is preferably 0.1 to 5 wt %, more preferably 0.2 to 3 wt %, and yet more preferably 0.3 to 1.5 wt %. The pH of the non-magnetic powder is 2 to 11, and is particularly preferably in the range of 5.5 to 10. The specific surface area of the non-magnetic powder is preferably 1 to 100 m²/g, more preferably 5 to 80 m²/g, and yet more preferably 10 to 70 m²/g. The crystallite size of the non-magnetic powder is preferably 0.004 to 1 μm, and more preferably 0.04 to 0.1 μm. The oil absorption measured using DBP is preferably 5 to 100 mL/100 g, more preferably 10 to 80 mL/100 g, and yet more preferably 20 to 60 mL/100 g. The specific gravity is preferably 1 to 12, and more preferably 3 to 6. The form may be any one of acicular, spherical, polyhedral, and tabular.

The ignition loss is preferably 20 wt % or less, and it is most preferable that there is no ignition loss. The Mohs hardness of the non-magnetic powder used in the present invention is preferably at least 4 but no greater than 10. The roughness factor of the surface of the powder is preferably 0.8 to 1.5, and more preferably 0.9 to 1.2. The amount of SA (stearic acid) absorbed by the non-magnetic powder is preferably 1 to 20 μmol/m², more preferably 2 to 15 μmol/m², and yet more preferably 3 to 8 μmol/m². The heat of wetting of the non-magnetic powder in water at 25° C. is preferably in the range of 200 to 600 erg/cm² (20 to 60 μJ/cm²). A solvent that gives a heat of wetting in this range can be used. The pH is preferably between 3 and 6. Water-soluble Na in the non-magnetic powder is preferably 0 to 150 ppm, and water-soluble Ca is preferably 0 to 50 ppm.

The surface of the non-magnetic powder is preferably subjected to a surface treatment so that Al₂O₃, SiO₂, TiO₂, ZrO₂, SnO₂, Sb₂O₃, ZnO, or Y₂O₃ is present. In terms of dispersibility in particular, Al₂O₃, SiO₂, TiO₂, and ZrO₂ are preferable, and Al₂O₃, SiO₂, and ZrO₂ are more preferable. They may be used in combination or singly. Depending on the intended purpose, a surface-treated layer may be obtained by co-precipitation, or a method in which it is firstly treated with alumina and the surface thereof is then treated with silica, or vice versa, can be employed. The surface-treated layer may be formed as a porous layer depending on the intended purpose, but it is generally preferable for it to be uniform and dense.

Specific examples of the non-magnetic powder used in the lower coated layer of the magnetic recording medium of the present invention include Nanotite (manufactured by Showa Denko K.K.), HIT-100 and ZA-G1 (manufactured by Sumitomo Chemical Co., Ltd.), α-hematite DPN-250, DPN-250BX, DPN-245, DPN-270BX, DBN-SA1, and DBN-SA3 (manufactured by Toda Kogyo Corp.), titanium oxide TTO-51 B, TTO-55A, TTO-55B, TTO-55C, TTO-55S, TTO-55D, and SN-100, and α-hematite E270, E271, E300, and E303 (manufactured by Ishihara Sangyo Kaisha Ltd.), titanium oxide STT-4D, STT-30D, STT-30, and STT-65C, and α-hematite α-40 (manufactured by Titan Kogyo Kabushiki Kaisha), MT-100S, MT-100T, MT-150W, MT-500B, MT-600B, MT-100F, and MT-500HD (manufactured by Tayca Corporation), FINEX-25, BF-1, BF-10, BF-20, and ST-M (manufactured by Sakai Chemical Industry Co., Ltd.), DEFIC-Y and DEFIC-R (manufactured by Dowa Mining Co., Ltd.), AS2BM and TiO₂P25 (manufactured by Nippon Aerosil Co., Ltd.), 100A and 500A (manufactured by Ube Industries, Ltd.), and calcined products thereof.

Particularly preferred non-magnetic powders are titanium dioxide and α-iron oxide.

α-Iron oxide (hematite) is employed under the various conditions below. That is, with regard to the α-Fe₂O₃ powder that can be used in the present invention, its precursor particles are acicular goethite particles obtained by, for example, a normal method (1) for forming acicular goethite particles in which a ferrous hydroxide colloid-containing suspension obtained by adding at least an equivalent amount of an aqueous solution of an alkali hydroxide to an aqueous ferrous solution is subjected to an oxidation reaction at a pH of 11 or higher at a temperature of 80° C. or less while passing an oxygen-containing gas therethrough, a method (2) for forming spindle-shaped goethite particles in which an oxidation reaction is carried out by passing an oxygen-containing gas into a suspension containing FeCO₃ obtained by reacting an aqueous solution of a ferrous salt and an aqueous solution of an alkali carbonate, a method (3) for forming acicular goethite nuclei particles by carrying out an oxidation reaction by passing an oxygen-containing gas into an aqueous solution of a ferrous salt containing a ferrous hydroxide colloid obtained by adding less than an equivalent amount of an aqueous solution of an alkali hydroxide or an alkali carbonate to an aqueous solution of a ferrous salt, and subsequently growing the acicular goethite nuclei particles by adding an aqueous solution of an alkali hydroxide to the aqueous solution of the ferrous salt containing the acicular goethite nuclei particles in an amount that is at least equivalent to the Fe²⁺ in the aqueous solution of the ferrous salt, and then passing through an oxygen-containing gas, and a method (4) for forming acicular goethite nuclei particles by carrying out an oxidation reaction by passing an oxygen-containing gas into an aqueous solution of a ferrous salt containing a ferrous hydroxide colloid obtained by adding less than an equivalent amount of an aqueous solution of an alkali hydroxide or an alkali carbonate to an aqueous ferrous solution, and subsequently growing the acicular goethite nuclei particles in an acidic to neutral region.

During the reaction to form goethite particles, different types of elements such as Ni, Zn, P, and Si, which are normally added in order to improve the characteristics of the powder, etc., may be added without any problem. The acicular goethite particles, which are the precursor particles, are dehydrated at a temperature in the range of 200° C. to 500° C., and if necessary further annealed by heating at a temperature in the range of 350° C. to 800° C. to give acicular α-Fe₂O₃ particles. An anti-sintering agent such as P, Si, B, Zr, or Sb can be attached without problem to the surface of the acicular goethite particles that are to be dehydrated or annealed. Annealing by heating at a temperature in the range of 350° C. to 800° C. is carried out for blocking pores formed on the surface of the dehydrated acicular α-Fe₂O₃ particles by melting the very surface of the particles, thus giving a smooth surface configuration, which is preferable.

The α-Fe₂O₃ powder used in the present invention is obtained by subjecting the dehydrated or annealed acicular α-Fe₂O₃ particles to dispersion in an aqueous solution to give a suspension, coating the surface of the α-Fe₂O₃ particles with an Al compound by adding the compound and adjusting the pH, and further subjecting the particles to filtration, washing with water, drying, grinding, and if necessary further degassing/compacting, etc.

As the Al compound used, an aluminum salt such as aluminum acetate, aluminum sulfate, aluminum chloride, or aluminum nitrate or an alkali aluminate such as sodium aluminate can be used.

In this case, the amount of Al compound added on an Al basis is preferably 0.01 to 50 wt % relative to the α-Fe₂O₃ powder. When the amount of Al compound added is in the above-mentioned range, the dispersibility thereof in a binder resin is sufficient, there are few Al compounds suspended on the particle surface, and Al compounds do not interact, which is preferable.

With regard to the non-magnetic powder of the lower layer in the present invention, the coating can be carried out using, in addition to the Al compound, one or more types of compounds chosen from an Si compound, and P, Ti, Mn, Ni, Zn, Zr, Sn, and Sb compounds. The amount of these compounds, which are used together with the Al compound, is preferably in the range of 0.01 to 50 wt % relative to the α-Fe₂O₃ powder. When the amount added is in the above-mentioned range, the effect of improving the dispersibility by the addition is sufficient, there are few suspended compounds that are not on the particle surface, and the compounds do not interact, which is preferable.

Methods for producing titanium dioxide are as follows. The main methods for producing titanium oxide are a sulfuric acid method and a chlorine method. In the sulfuric acid method, an ilmenite ore is digested with sulfuric acid, and Ti, Fe, etc. are extracted as sulfates. Iron sulfate is removed by crystallization, and the remaining titanyl sulfate solution is purified by filtration and then subjected to thermal hydrolysis so as to precipitate hydrated titanium oxide. After this is filtered and washed, impurities are removed by washing, a particle size regulator, etc. is added thereto, and the mixture is calcined at 80° C. to 1,000° C. to give crude titanium oxide. The rutile type and the anatase type can be separated according to the type of a nucleating agent that is added when carrying out hydrolysis. This crude titanium oxide is subjected to grinding, size adjustment, surface treatment, etc. As an ore for the chlorine method, natural rutile or synthetic rutile is used. The ore is chlorinated at high temperature under reducing conditions, Ti is converted into TiCl₄ and Fe is converted into FeCl₂, and iron oxide solidifies by cooling and is separated from liquid TiCl₄. The crude TiCl₄ thus obtained is purified by distillation, then a nucleating agent is added, and the mixture is reacted momentarily with oxygen at a temperature of 1,000° C. or higher to give crude titanium oxide. A finishing method for imparting pigmentary properties to the crude titanium oxide formed by this oxidative decomposition process is the same as that for the sulfuric acid method.

The surface treatment is carried out by dry-grinding the above-mentioned titanium oxide material, then adding water and a dispersant thereto, and subjecting it to rough classification by wet-grinding and centrifugation. Subsequently, the fine grain slurry is transferred to a surface treatment vessel, and here surface coating with a metal hydroxide is carried out. Firstly, a predetermined amount of an aqueous solution of a salt such as Al, Si, Ti, Zr, Sb, Sn, or Zn is added, an acid or an alkali for neutralizing this is added, and the hydrated oxide thus formed is used for coating the surface of the titanium oxide particles. Water-soluble salts produced as a by-product are removed by decantation, filtration, and washing. Finally the pH of the slurry is adjusted, and it is filtered and washed with pure water. The cake thus washed is dried by a spray dryer or a band dryer. This dried product is ground using a jet mill to give a final product.

In addition to the an aqueous system, it is also possible to expose a titanium oxide powder to AlCl₃ or SiCl₄ vapor and then to steam, thereby carrying out a surface treatment with Al or Si. Other methods for preparing a pigment can be referred to in G. D. Parfitt and K. S. W. Sing, ‘Characterization of Powder Surfaces’ Academic Press, 1976.

Incorporation of carbon black into the lower coated layer can give the known effects of a lowering of surface electrical resistance (Rs), a reduction in light transmittance, and giving a desired micro Vickers hardness. The presence of carbon black in the lower layer can exhibit an effect of storing a lubricant. Types of carbon black that can be used include furnace black for rubber, thermal black for rubber, black for coloring, and acetylene black. The carbon black used in the lower layer should have characteristics that have been optimized as follows according to a desired effect, and the effect can be increased by the use thereof in combination.

The specific surface area of the carbon black in the lower layer is preferably 100 to 500 m²/g, and more preferably 150 to 400 m²/g, and the DBP oil absorption thereof is preferably 20 to 400 mL/100 g, and more preferably 30 to 200 mL/100 g. The particle size of the carbon black is preferably 5 to 80 nm, more preferably 10 to 50 nm, and yet more preferably 10 to 40 nm. The pH of the carbon black is preferably 2 to 10, the water content is preferably 0.1% to 10%, and the tap density is preferably 0.1 to 1 g/mL.

Specific examples of the carbon black used in the present invention include BLACKPEARLS 2000, 1300, 1000, 900, 800, 880, and 700, and VULCAN XC-72 (manufactured by Cabot Corporation), #3050B, #3150B, #3250B, #3750B, #3950B, #950, #650B, #970B, #850B, MA-600, MA-230, #4000, and #4010 (manufactured by Mitsubishi Chemical Corporation), CONDUCTEX SC, and RAVEN 8800, 8000, 7000, 5750, 5250, 3500, 2100, 2000, 1800, 1500, 1255, and 1250 (manufactured by Columbia Carbon Co.), Ketjen Black EC (manufactured by Akzo), and Ketjen Black EC (manufactured by Ketjen Black International Company Ltd.).

The carbon black may be subjected to any of a surface treatment with a dispersant, etc., grafting with a resin, or a partial surface graphitization. The carbon black may also be dispersed in a binder prior to addition to a coating solution. The carbon black can be preferably used in a range not exceeding 50 wt % relative to the above-mentioned inorganic powder, and in a range not exceeding 40 wt % relative to the total weight of the non-magnetic layer. The carbon black can be used singly or in a combination of different types thereof. The carbon black that can be used in the present invention can be referred to in, for example, the ‘Kabon Burakku Handobukku’ (Carbon Black Handbook) (edited by the Carbon Black Association of Japan).

It is also possible to add an organic powder to the lower coated layer depending on the intended purpose. Examples thereof include an acrylic styrene resin powder, a benzoguanamine resin powder, a melamine resin powder, and a phthalocyanine pigment, but a polyolefin resin powder, a polyester resin powder, a polyamide resin powder, a polyimide resin powder, and a polyfluoroethylene resin can also be used. Production methods such as those described in JP-A-62-18564 and JP-A-60-255827 can be used.

The binder, the lubricant, the dispersant, the additive, the solvent, the dispersion method, etc. for the lower coated layer may employ those used for the magnetic layer. In particular, with regard to the amount and type of binder, the additive, and the amount and type of dispersant, known techniques for the magnetic layer may be applied.

III. Non-Magnetic Support

With regard to the non-magnetic support that can be used in the present invention, known biaxially stretched films such as polyethylene terephthalate, polyethylene naphthalate, polyamide, polyamideimide, and aromatic polyamide can be used. Polyethylene terephthalate, polyethylene naphthalate, and polyamide are preferred.

These supports can be subjected in advance to a corona discharge treatment, a plasma treatment, a treatment for enhancing adhesion, a thermal treatment, etc. The non-magnetic support that can be used in the present invention preferably has a surface roughness such that its center plane average surface roughness Ra is in the range of 3 to 10 nm for a cutoff value of 0.25 mm.

IV. Smoothing Layer

The magnetic recording medium of the present invention may be provided with a smoothing layer. The smoothing layer referred to here is a layer for burying protrusions on the surface of the non-magnetic support; it is provided between the non-magnetic support and the magnetic layer when the magnetic recording medium is provided with the magnetic layer above the non-magnetic support, and it is provided between the non-magnetic support and the non-magnetic layer when the magnetic recording medium is provided with the non-magnetic layer and the magnetic layer in that order above the non-magnetic support.

The smoothing layer can be formed by curing a radiation curing type compound by exposure to radiation. The radiation curing type compound referred to here is a compound having the property of polymerizing or crosslinking when irradiated with radiation such as ultraviolet rays or an electron beam, thus increasing the molecular weight and carrying out curing.

V. Backcoat Layer

In general, there is a strong requirement for magnetic tapes for recording computer data to have better repetitive transport properties than video tapes and audio tapes. In order to maintain such high storage stability, a backcoat layer can be provided on the surface of the non-magnetic support opposite to the surface where the non-magnetic layer and the magnetic layer are provided. As a coating solution for the backcoat layer, a binder and a particulate component such as an abrasive or an antistatic agent are dispersed in an organic solvent. As a granular component, various types of inorganic pigment or carbon black can be used. As the binder, a resin such as nitrocellulose, a phenoxy resin, a vinyl chloride resin, or a polyurethane can be used singly or in combination.

VI. Layer Structure

In the constitution of the magnetic recording medium used in the present invention, the thickness of the non-magnetic support is preferably 3 to 80 μm. When the smoothing layer is provided between the non-magnetic support and the non-magnetic layer or the magnetic layer, the thickness of the smoothing layer is preferably 0.01 to 0.8 μm, and more preferably 0.02 to 0.6 μm. The thickness of the backcoat layer provided on the surface of the non-magnetic support opposite to the surface where the non-magnetic layer and the magnetic layer are provided is preferably 0.1 to 1.0 μm, and more preferably 0.2 to 0.8 μm.

The thickness of the magnetic layer is optimized according to the saturation magnetization and the head gap of the magnetic head and the bandwidth of the recording signal, but it is preferably 0.01 to 0.5 μm, more preferably 0.02 to 0.3 μm, and yet more preferably 0.03 to 0.2 μm. The percentage variation in thickness of the magnetic layer is preferably ±50% or less, and more preferably ±40% or less. The magnetic layer can be at least one layer, but it is also possible to provide two or more separate layers having different magnetic properties, and a known configuration for a multilayer magnetic layer can be employed.

The thickness of the non-magnetic layer is preferably 0.2 to 3.0 μm, more preferably 0.3 to 2.5 μm, and yet more preferably 0.4 to 2.0 μm. The non-magnetic layer of the magnetic recording medium of the present invention exhibits its effect if it is substantially non-magnetic, but even if it contains a small amount of a magnetic substance as an impurity or intentionally, if the effects of the present invention are exhibited the constitution can be considered to be substantially the same as that of the magnetic recording medium of the present invention. ‘Substantially the same’ referred to here means that the non-magnetic layer has a residual magnetic flux density of 10 mT (100 G) or less or a coercive force of 7.96 kA/m (100 Oe) or less, and preferably has no residual magnetic flux density and no coercive force.

VII. Production Method

A process for producing a magnetic layer coating solution for the magnetic recording medium used in the present invention comprises at least a kneading step, a dispersing step and, optionally, a blending step that is carried out prior to and/or subsequent to the above-mentioned steps. Each of these steps may be composed of two or more separate stages. All materials, including the ferromagnetic hexagonal ferrite powder, the ferromagnetic metal powder, the non-magnetic powder, the binder, the carbon black, the abrasive, the antistatic agent, the lubricant, and the solvent used in the present invention may be added in any step from the beginning or during the course of the step. The addition of each material may be divided across two or more steps. For example, a polyurethane can be divided and added in a kneading step, a dispersing step, and a blending step for adjusting the viscosity after dispersion. To attain the object of the present invention, a conventionally known production technique may be employed as a part of the steps. In the kneading step, it is preferable to use a powerful kneading machine such as an open kneader, a continuous kneader, a pressure kneader, or an extruder. When a kneader is used, all or a part of the binder (preferably 30 wt % or above of the entire binder) is preferably kneaded with the magnetic powder or the non-magnetic powder at 15 to 500 parts by weight of the binder relative to 100 parts by weight of the magnetic substance. Details of these kneading treatments are described in JP-A-1-106338 and JP-A-1-79274. For the dispersion of the magnetic layer solution and a non-magnetic layer solution, glass beads can be used. As such glass beads, a dispersing medium having a high specific gravity such as zirconia beads, titania beads, or steel beads is suitably used. An optimal particle size and packing density of these dispersing media is used. A known disperser can be used.

The process for producing the magnetic recording medium of the present invention includes, for example, coating the surface of a moving non-magnetic support with a magnetic layer coating solution so as to give a predetermined coating thickness. A plurality of magnetic layer coating solutions can be applied successively or simultaneously in multilayer coating, and a lower magnetic layer coating solution and an upper magnetic layer coating solution can also be applied successively or simultaneously in multilayer coating. As coating equipment for applying the above-mentioned magnetic layer coating solution or the lower magnetic layer coating solution, an air doctor coater, a blade coater, a rod coater, an extrusion coater, an air knife coater, a squeegee coater, a dip coater, a reverse roll coater, a transfer roll coater, a gravure coater, a kiss coater, a cast coater, a spray coater, a spin coater, etc. can be used. With regard to these, for example, ‘Saishin Kotingu Gijutsu’ (Latest Coating Technology) (May 31, 1983) published by Sogo Gijutsu Center can be referred to.

In the case of a magnetic tape, the coated layer of the magnetic layer coating solution is subjected to a magnetic field alignment treatment in which the ferromagnetic powder contained in the coated layer of the magnetic layer coating solution is aligned in the longitudinal direction using a cobalt magnet or a solenoid. In the case of a disk, although sufficient isotropic alignment can sometimes be obtained without using an alignment device, it is preferable to employ a known random alignment device such as, for example, arranging obliquely alternating cobalt magnets or applying an alternating magnetic field with a solenoid. The isotropic alignment referred to here means that, in the case of a ferromagnetic metal powder, in general, in-plane two-dimensional random is preferable, but it can be three-dimensional random by introducing a vertical component. In the case of a ferromagnetic hexagonal ferrite powder, in general, it tends to be in-plane and vertical three-dimensional random, but in-plane two-dimensional random is also possible. By using a known method such as magnets having different poles facing each other so as to make vertical alignment, circumferentially isotropic magnetic properties can be introduced. In particular, when carrying out high density recording, vertical alignment is preferable. Furthermore, circumferential alignment may be employed using spin coating.

It is preferable for the drying position for the coating to be controlled by controlling the drying temperature and blowing rate and the coating speed; it is preferable for the coating speed to be 20 to 1,000 m/min and the temperature of drying air to be 60° C. or higher, and an appropriate level of pre-drying may be carried out prior to entering a magnet zone.

After drying is carried out, the coated layer is subjected to a surface smoothing treatment. The surface smoothing treatment employs, for example, super calender rolls, etc. By carrying out the surface smoothing treatment, cavities formed by removal of the solvent during drying are eliminated, thereby increasing the packing ratio of the ferromagnetic powder in the magnetic layer, and a magnetic recording medium having high electromagnetic conversion characteristics can thus be obtained.

With regard to calendering rolls, rolls of a heat-resistant plastic such as epoxy, polyimide, polyamide, or polyamideimide are used. It is also possible to carry out a treatment with metal rolls. The magnetic recording medium of the present invention preferably has a surface center plane average roughness in the range of 0.1 to 4.0 nm for a cutoff value of 0.25 mm, and more preferably 0.5 to 3.0 nm, which is extremely smooth. As a method therefor, a magnetic layer formed by selecting a specific ferromagnetic powder and binder as described above is subjected to the above-mentioned calendering treatment. With regard to calendering conditions, the calender roll temperature is preferably in the range of 60° C. to 100° C., more preferably in the range of 70° C. to 100° C., and particularly preferably in the range of 80° C. to 100° C., and the pressure is preferably in the range of 100 to 500 kg/cm, more preferably in the range of 200 to 450 kg/cm, and particularly preferably in the range of 300 to 400 kg/cm.

As thermal shrinkage reducing means, there is a method in which a web is thermally treated while handling it with low tension, and a method (thermal treatment) involving thermal treatment of a tape when it is in a layered configuration such as in bulk or installed in a cassette, and either can be used. In the former method, the effect of the imprint of protrusions of the surface of the backcoat layer is small, but the thermal shrinkage cannot be greatly reduced. On the other hand, the latter thermal treatment can improve the thermal shrinkage greatly, but since the effect of the imprint of protrusions of the surface of the backcoat layer is strong, the surface of the magnetic layer is roughened, and this causes the output to decrease and the noise to increase. In particular, a high output and low noise magnetic recording medium can be provided for the magnetic recording medium accompanying the thermal treatment. The magnetic recording medium thus obtained can be cut to a desired size using a cutter, a stamper, etc. before use.

VIII. Physical Properties

The saturation magnetic flux density of the magnetic layer of the magnetic recording medium of the present invention is preferably 100 to 300 mT (1,000 to 3,000 G). The coercive force (Hc) of the magnetic layer is preferably 143.3 to 318.4 kA/m (1,800 to 4,000 Oe), and more preferably 159.2 to 278.6 kA/m (2,000 to 3,500 Oe). It is preferable for the coercive force distribution to be narrow, and the SFD and SFDr are preferably 0.6 or less, and more preferably 0.2 or less.

The coefficient of friction, with respect to a head, of the magnetic recording medium used in the present invention is preferably 0.5 or less at a temperature of −10° C. to 40° C. and a humidity of 0% to 95%, and more preferably 0.3 or less. The electrostatic potential is preferably −500 V to +500 V. The modulus of elasticity of the magnetic layer at an elongation of 0.5% is preferably 0.98 to 19.6 GPa (100 to 2,000 kg/mm²) in each direction within the plane, and the breaking strength is preferably 98 to 686 MPa (10 to 70 kg/mm²); the modulus of elasticity of the magnetic recording medium is preferably 0.98 to 14.7 GPa (100 to 1,500 kg/mm²) in each direction within the plane, the residual elongation is preferably 0.5% or less, and the thermal shrinkage at any temperature up to and including 100° C. is preferably 1% or less, more preferably 0.5% or less, and most preferably 0.1% or less.

The glass transition temperature of the magnetic layer (the maximum point of the loss modulus in a dynamic viscoelasticity measurement at 110 Hz) is preferably 50° C. to 180° C., and that of the non-magnetic layer is preferably 0C to 180° C. The loss modulus is preferably in the range of 1×10⁷ to 8×10⁸ Pa (1×10⁸ to 8×10⁹ dyne/cm²), and the loss tangent is preferably 0.2 or less. When the loss tangent is 0.2 or less, the problem of tackiness is suppressed. These thermal properties and mechanical properties are preferably substantially identical to within 10% in each direction in the plane of the medium.

Residual solvent in the magnetic layer is preferably 100 mg/m² or less, and more preferably 10 mg/m² or less. The porosity of the coating layer is preferably 30 vol % or less for both the non-magnetic layer and the magnetic layer, and more preferably 20 vol % or less. In order to achieve a high output, the porosity is preferably low, but there are cases in which a certain value should be maintained depending on the intended purpose. For example, in the case of disk media where repetitive use is considered to be important, a high porosity is often preferable from the point of view of storage stability.

The center plane surface roughness Ra of the magnetic layer is preferably 4.0 nm or less, more preferably 3.0 nm or less, and yet more preferably 2.0 nm or less, when measured using a TOPO-3D digital optical profiler (manufactured by Wyko Corporation). The maximum height SRₘₐₓ of the magnetic layer is preferably 0.5 μm or less, the ten-point average roughness SRz is 0.3 μm or less, the center plane peak height SRp is 0.3 μm or less, the center plane valley depth SRv is 0.3 μm or less, the center plane area factor SSr is 20% to 80%, and the average wavelength Sλa is 5 to 300 μm. It is possible to set the number of surface protrusions on the magnetic layer having a size of 0.01 to 1 μm at any level in the range of 0 to 2,000 protrusions per 100 μm, and by so doing the electromagnetic conversion characteristics and the coefficient of friction can be optimized, which is preferable. They can be controlled easily by controlling the surface properties of the support by means of a filler, the particle size and the amount of a powder added to the magnetic layer, and the shape of the roll surface in the calendering process. The curl is preferably within ±3 mm.

When the magnetic recording medium of the present invention has a non-magnetic layer and a magnetic layer, it can easily be anticipated that the physical properties of the non-magnetic layer and the magnetic layer can be varied according to the intended purpose. For example, the elastic modulus of the magnetic layer can be made high, thereby improving the storage stability, and at the same time the elastic modulus of the non-magnetic layer can be made lower than that of the magnetic layer, thereby improving the head contact of the magnetic recording medium.

A head used for playback of signals recorded magnetically on the magnetic recording medium of the present invention is not particularly limited, but an MR head is preferably used. When an MR head is used for playback of the magnetic recording medium of the present invention, the MR head is not particularly limited and, for example, a GMR head or a TMR head can be used. A head used for magnetic recording is not particularly limited, but it is preferable for the saturation magnetization to be 1.0 T or more, and preferably 1.5 T or more.

In accordance with the present invention, it is possible to provide a production process for a carbonic acid ester, the process enabling a high-purity carbonic acid ester to be obtained simply, and to provide a carbonic acid ester obtained by the production process.

Furthermore, it is possible to provide a magnetic recording medium having excellent electromagnetic conversion characteristics, durability, and storage stability by the use of the carbonic acid ester.

### EXAMPLES

The present invention is explained more specifically below by reference to Examples, but the present invention should not be construed as being limited to the Examples. ‘Parts’ in the Examples means ‘parts by weight’ unless otherwise specified.

### Example 1

### Lubricant A Synthetic Example

A flask was charged with 108.2 parts of 1-octadecanol, 290 parts of hexane, and 35 parts of pyridine, and cooled while stirring. 42 parts of 2-ethylhexyl chloroformate was further added dropwise to this flask while cooling and stirring over 2 hours. While further stirring the interior of the flask, it was taken out to room temperature and allowed to stand for 6 hours. Water was added to this reaction mixture, the mixture was stirred and then left to stand, and the aqueous layer was run off using a separatory funnel. Methanol was added, the mixture was stirred and then left to stand, and the methanol phase was separated; this operation was repeated three times. The remaining hexane solution was concentrated under vacuum, and about 93 parts of lubricant A, which was a colorless transparent liquid, was obtained.

This liquid was diluted 10 times with ethyl acetate and 1 μL thereof was separated by means of thin layer chromatography, but 1-octadecanol was not detected.

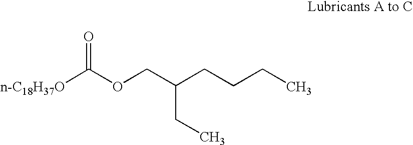

### Example 2

### Lubricant B Synthetic Example

The procedure of Example 1 was repeated except that the methanol was changed to acetonitrile, and about 93 parts of lubricant B, which was a colorless transparent liquid, was obtained.

This liquid was diluted 10 times with ethyl acetate and 1 μL thereof was separated by means of thin layer chromatography, but 1-octadecanol was not detected.

### Comparative Example 1

### Lubricant C Synthetic Example

The procedure of Example 1 was repeated except that the methanol was changed to water; when a hexane solution from which the aqueous phase had been run off was concentrated under vacuum, a large number of crystalline components were precipitated at room temperature, and lubricant C was thus obtained.

This was again diluted 10 times with hexane and 1 μL thereof was separated by means of thin layer chromatography, and 1-octadecanol was detected.

### Examples 3 and 4

### Lubricants D and E Synthetic Examples

The extraction procedure of Example 1 was repeated except that the 1-octadecanol and 2-ethylhexyl chloroformate of Example 1 were changed respectively to alcohols and chloroformate esters having the structures for R¹ and R² shown in Table 1, and lubricants D and E were obtained.

The lubricants D and E thus obtained were diluted 10 times with ethyl acetate and 1 μL thereof was separated by means of thin layer chromatography, but the corresponding alcohols were not detected.

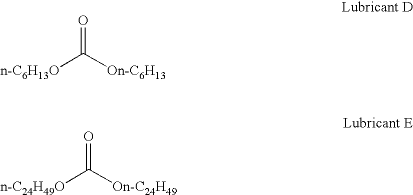

### Synthetic Examples 1 and 2

The extraction procedure of Example 1 was repeated except that the 1-octadecanol and 2-ethylhexyl chloroformate of Example 1 were changed respectively to alcohols and chloroformate esters having the structures for R¹ and R² shown in Table 1, and lubricants F and G were obtained.

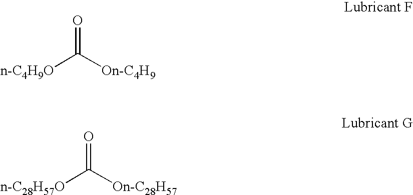

### Synthetic Example 3

Synthesis of Lubricant H (2-ethylhexyl nonadecanoate)

0.1 parts by weight of p-toluenesulfonic acid monohydrate was added to 29.8 parts by weight of nonadecanoic acid, 19.5 parts by weight of 2-ethyl-1-hexanol, and 86.5 parts by weight of toluene, the mixture was refluxed by heating for 4 hours while stirring, and toluene was then distilled off in this state. This reaction mixture was subjected to vacuum distillation, and lubricant H, which is a fatty acid ester, was obtained.

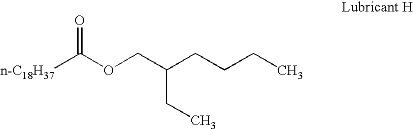

### Synthetic Example 4

The extraction procedure of Example 1 was repeated except that the 1-octadecanol and 2-ethylhexyl chloroformate of Example 1 were changed respectively to the alcohol and the chloroformate ester having the structures for R¹ and R² shown in Table 1, and lubricant I was obtained.

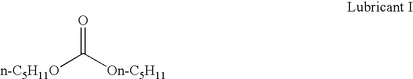

### Examples 5 to 10 and Comparative Examples 2 to 9

Preparation of Upper Layer Magnetic Solution

100 parts of a ferromagnetic metal powder (Co/Fe=30 atom %, Hc: 2,350 Oe (187 kA/m), S_{BET}: 55 m²/g, surface treated with Al₂O₃, SiO₂, and Y₂O₃, average major axis length: 50 nm, average acicular ratio: 7, σs: 120 A·m²/kg) was ground in an open kneader for 10 minutes, subsequently

| | | |
|---|---|---|
| carbon black (average particle size 80 nm) | 2 | parts |
| a vinyl chloride resin (MR-110, manufac- | 10 | parts |
| tured by Nippon Zeon Corporation) | | |
| a polyester polyurethane (UR8300, | 6 | parts (solids |
| manufactured by Toyobo Co., Ltd.) | | content), and |
| methyl ethyl ketone/cyclohexanone = 1/1 | 60 | parts |

were added thereto, and the mixture was kneaded for 60 minutes. To this
mixture,

| | | |
|---|---|---|
| methyl ethyl ketone/cyclohexanone = 1/1 | 200 | parts |

was added over 6 hours while operating the open kneader. Subsequently,

| | | |
|---|---|---|
| an α-Al₂O₃ dispersion | 20 | parts |

was added thereto, and the mixture was dispersed in a sand grinder for
120 minutes. Furthermore,

| | | |
|---|---|---|
| a polyisocyanate | 4 | parts |
| (Coronate 3041, manufactured by Nippon | | (solids content) |
| Polyurethane Industry Co., Ltd.) | | |
| stearic acid | 1 | part |
| a lubricant described in Table 1 below | 2 | parts |
| stearamide | 0.2 | parts, and |
| toluene | 50 | parts |

were added thereto, and the mixture was stirred and mixed for 20 minutes.
Following this, the mixture was filtered using a filter having an average
pore size of 1 μm to give a magnetic coating solution.

In order to change H₁₅/H₁₀, the α-Al₂O₃ dispersion was changed in the range of 0 to 30 parts.

Preparation of Lower Layer Non-Magnetic Solution

85 parts of titanium oxide (average particle size 0.035 μm, rutile crystal type, TiO₂ content 90% or greater, surface treated with alumina, S_{BET} 35 to 42 m²/g, true specific gravity 4.1, pH 6.5 to 8.0) and 15 parts of carbon black (Ketjen black EC, manufactured by Ketjen Black International Company Ltd.) were ground in an open kneader for 10 minutes, subsequently 17 parts of a vinyl chloride copolymer (MR-110, manufactured by Nippon Zeon Corporation), 10 parts (solids content) of a sulfonic acid-containing polyurethane resin (UR8200, manufactured by Toyobo Co., Ltd.), and 60 parts of cyclohexanone were added thereto, and the mixture was kneaded for 60 minutes. Subsequently,

| | | |
|---|---|---|
| methyl ethyl ketone/cyclohexanone = 6/4 | 200 | parts |

was added thereto, and the mixture was dispersed in a sand mill for
120 minutes. To this were added

| | | |
|---|---|---|
| a polyisocyanate | 5 | parts |
| (Coronate 3041, manufactured by | | (solids content) |
| Nippon Polyurethane Industry Co., Ltd.) | | |
| stearic acid | 1 | part |
| a lubricant described in Table 1 below | 2 | parts |
| oleic acid | 1 | part, and |
| methyl ethyl ketone | 50 | parts, |

and the mixture was stirred and mixed for 20 minutes, then filtered
using a filter having an average pore size of 1 μm to give a non-magnetic
coating solution.

The surface of a 62 μm thick polyethylene terephthalate support was coated with the non-magnetic coating solution thus obtained and, immediately after that, with the magnetic coating solution by simultaneous multilayer coating so that the dry thicknesses thereof were 1.5 μm and 0.2 μm respectively. Before the magnetic coating solution had dried, it was subjected to magnetic field alignment using a 5,000 G Co magnet and a 4,000 G solenoid magnet, and after removing the solvent by drying, it was subjected to a calender treatment employing a metal roll-metal roll-metal roll-metal roll-metal roll-metal roll-metal roll combination (speed 100 m/min, line pressure 300 kg/cm, temperature 90° C.) and then slit to a width of ½ inch (12.65 mm).

Measurement Methods

1. Height Distribution of Protrusions on Surface of Magnetic Layer

The height distribution of protrusions was measured using an atomic force microscope (Nanoscope AFM, manufactured by Digital Instruments). Measurement was carried out using a regular tetrahedral contact mode probe with a tip half angle of 350 and a radius of curvature of 100 nm or below using Ver. 3.25 software. The test sample was a 15 μm×15 μm square, and the measurement result was corrected for inclination, etc. by a third-order correction, and processed using a command for obtaining the number of peaks in a Roughness Analysis to give the protrusion distribution.

2. Electromagnetic Conversion Characteristics

Measurement was carried out by mounting a recording head (MIG, gap 0.15 μm, 1.8 T) and an MR playback head on a drum tester. The playback output was measured at a speed of the medium relative to the head of 1 to 3 m/min and a surface recording density of 0.57 Gbit/(inch)² (0.89 Mbit/mm²) and expressed as a relative value where the playback output of Comparative Example 2 was 0 dB.

3. Durability and Storage Stability

The sliding durability of the tape was measured as follows. That is, the tape was made to slide at a sliding speed of 2 m/sec repeatedly for 10,000 passes under an environment of 40° C. and 10% RH with the magnetic layer surface in contact with an AlTiC cylindrical rod at a load of 100 g (T1), and tape damage was then evaluated using the rankings below.

Furthermore, 600 m of a tape was stored at 60° C. and 90% RH for 6 months while wound on a reel for an LTO-G3 cartridge. The tape after storage was evaluated in the same manner.

Excellent: slightly scratched, but area without scratches was larger.

Good: area with scratches was larger than area without scratches.

Poor: magnetic layer completely peeled off.

Evaluation results for Examples 5 to 10 and Comparative Examples 2 to 9 are given in Table 1 below.

TABLE 1

| | Lubricant | | | | | | | | | | |
|---|---|---|---|---|---|---|---|---|---|---|---|
| | | | | Molecular structure | | | | | | | |
| | | | | | | Total | Upper | | | Durability, storage | |
| | | | | Number | Number | number of | layer | | Electromagnetic | stability | |
| | | | | of | of | carbons | α-Al₂O₃ | | conversion | | After |
| | | | Extraction | carbons | carbons | of R¹ and | dispersion | | characteristics | Before | 60° C. 90% |
| | Lubricant | Type | solvent | of R¹ | of R² | R² | (parts) | H₁₅/H₁₀ | (dB) | storage | storage |
| Ex. 5 | A | Carbonic | Methanol | 18 | 8 | 26 | 20 | 0.16 | 2.2 | Excellent | Excellent |
| | | acid ester | | | | | | | | | |
| Ex. 6 | B | Carbonic | Acetonitrile | 18 | 8 | 26 | 20 | 0.16 | 2.2 | Excellent | Excellent |
| | | acid ester | | | | | | | | | |
| Ex. 7 | D | Carbonic | Methanol | 6 | 6 | 12 | 20 | 0.16 | 2.4 | Good | Good |
| | | acid ester | | | | | | | | | |
| Ex. 8 | E | Carbonic | Methanol | 24 | 24 | 48 | 20 | 0.16 | 2.1 | Good | Good |
| | | acid ester | | | | | | | | | |
| Ex. 9 | A | Carbonic | Methanol | 18 | 8 | 26 | 2 | 0.01 | 3.5 | Good | Good |
| | | acid ester | | | | | | | | | |
| Ex. 10 | A | Carbonic | Methanol | 18 | 8 | 26 | 30 | 0.19 | 1.6 | Excellent | Excellent |
| | | acid ester | | | | | | | | | |
| Comp. | C | Carbonic | Water | 18 | 8 | 26 | 30 | 0.24 | 0.0 | Poor | Poor |
| Ex. 2 | | acid ester | | | | | | | | | |
| Comp. | I | Carbonic | Methanol | 5 | 5 | 10 | 30 | 0.24 | 0.0 | Poor | Poor |
| Ex. 3 | | acid ester | | | | | | | | | |
| Comp. | F | Carbonic | Methanol | 4 | 4 | 8 | 30 | 0.24 | 0.0 | Poor | Poor |
| Ex. 4 | | acid ester | | | | | | | | | |
| Comp. | G | Carbonic | Methanol | 28 | 28 | 56 | 20 | 0.16 | 2.0 | Poor | Poor |
| Ex. 5 | | acid ester | | | | | | | | | |
| Comp. | A | Carbonic | Methanol | 18 | 8 | 26 | 0 | 0.00 | 3.6 | Poor | Poor |
| Ex. 6 | | acid ester | | | | | | | | | |
| Comp. | A | Carbonic | Methanol | 18 | 8 | 26 | 30 | 0.24 | 0.0 | Excellent | Excellent |
| Ex. 7 | | acid ester | | | | | | | | | |
| Comp. | I | Carbonic | Methanol | 5 | 5 | 10 | 20 | 0.16 | 2.1 | Poor | Poor |
| Ex. 8 | | acid ester | | | | | | | | | |
| Comp. | H | Fatty acid | — | 18 | 8 | 26 | 20 | 0.16 | 2.1 | Good | Poor |
| Ex. 9 | | ester | | | | | | | | | |

What is claimed is:

1. A process for producing a carbonic acid ester, the process comprising:
a step of synthesizing a saturated alkyl carbonic acid ester represented by Formula (1) so as to give the saturated alkyl carbonic acid ester represented by Formula (1) as a crude product; and
a step of subjecting the crude product to liquid-liquid extraction using a saturated hydrocarbon solvent and a solvent comprising an organic solvent that is not infinitely miscible with the saturated hydrocarbon solvent so as to give the saturated alkyl carbonic acid ester represented by Formula (1) as a purified product, 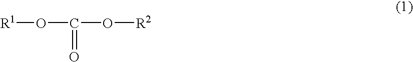
wherein R¹ and R² independently denote a saturated hydrocarbon group provided that the sum of the number of carbons in R¹ and the number of carbons in R² is at least 12 but no greater than 50.

2. The process for producing a carbonic acid ester according to claim 1, wherein the synthesis of the saturated alkyl carbonic acid ester represented by Formula (1) above is carried out by reacting a chloroformate ester and an alcohol.

3. The process for producing a carbonic acid ester according to claim 1, wherein the synthesis of the saturated alkyl carbonic acid ester represented by Formula (1) above is carried out using a catalyst.

4. The process for producing a carbonic acid ester according to claim 3, wherein the catalyst is an organic base having no N—H bond when neutral, or lithium hydroxide.

5. The process for producing a carbonic acid ester according to claim 1, wherein said R¹ and/or R² are straight-chain saturated hydrocarbon groups.

6. The process for producing a carbonic acid ester according to claim 5, wherein the straight-chain saturated hydrocarbon group is butyl, hexyl, octyl, decyl, dodecyl, tetradecyl, hexadecyl, octadecyl, eicosanyl, or docosanyl.

7. The process for producing a carbonic acid ester according to claim 1, wherein the saturated hydrocarbon solvent is heptane, hexane, decane, undecane, dodecane, cyclohexane, or a mixed solvent thereof.

8. The process for producing a carbonic acid ester according to claim 1, wherein the saturated hydrocarbon solvent is heptane or hexane.

9. The process for producing a carbonic acid ester according to claim 1, wherein the organic solvent that is not infinitely miscible with the saturated hydrocarbon solvent is methanol, ethanol, propanol, acetonitrile, or ethylene glycol and/or propylene glycol.

10. The process for producing a carbonic acid ester according to claim 1, wherein the organic solvent that is not infinitely miscible with the saturated hydrocarbon solvent is methanol or acetonitrile.

11. The process for producing a carbonic acid ester according to claim 1, wherein the saturated hydrocarbon solvent is heptane or hexane, and the organic solvent that is not infinitely miscible with the saturated hydrocarbon solvent is methanol or acetonitrile.

12. A carbonic acid ester produced by the production process according to claim 1.

13. A magnetic recording medium comprising:
a non-magnetic support and, above the non-magnetic support, a magnetic layer comprising a ferromagnetic powder dispersed in a binder, the magnetic layer comprising the carbonic acid ester according to claim 12 and having on the surface a number of protrusions that satisfies Formula (2),
0.01≦H₁₅/H₁₀≦0.20 (2)
wherein H₁₀ denotes the number of protrusions per unit area on the surface of the magnetic layer that have a height of less than 10 nm (number/μm²), and H₁₅ denotes the number of protrusions per unit area on the surface of the magnetic layer that have a height of 15 nm or greater (number/μm²).
